(19) United States
(12) Patent Application Publication
(10) Pub. No.: US 2024/0390631 A1
(43) Pub. Date: Nov. 28, 2024

(54) PATIENT INTERFACE DESIGN FOR ACOUSTIC DETECTION

(71) Applicant: ResMed Pty Ltd, Bella Vista (AU)

(72) Inventors: Liam Holley, Sydney (AU); Ellen Patricia GIBBS, Sydney (AU)

(73) Assignee: ResMed Pty Ltd, Bella Vista (AU)

(21) Appl. No.: 18/672,697

(22) Filed: May 23, 2024

Related U.S. Application Data
(60) Provisional application No. 63/504,298, filed on May 25, 2023.

(51) Int. Cl. A61M 16/08

(52) U.S. Cl. CPC A61M 16/0816; A61M 2205/42

(57) ABSTRACT

Respiratory patient interface (e.g., mask) structures may be modified to include acoustical feature(s) (AF) to provide differences in structure that can produce, when acoustically sensed, a unique acoustic signature for identification of a particular model mask and thereby differentiate it from other model masks. The added AF may be generally similar across different models of patient interface but have detectable differences between the models. However, the added AF of a particular model are typically substantially the same for all masks of that particular model. Such AF may, for example, be a change in material composition of a section or part of a flow path of the patient interface, a change in a dimension, such as a length, of a section or part of a flow path of the patient interface, and/or an expansion and/or contraction of a section (e.g., diameter) or part of a flow path of patient interface.

### CROSS REFERENCE TO RELATED APPLICATIONS

This application claims the benefit of U.S. Provisional Patent Application Ser. No. 63/504,298, filed 25 May 2023, the entire disclosure of which is hereby incorporated herein by reference.

### FIELD OF THE TECHNOLOGY

The present technology relates to therapy apparatus and identification of components of such devices, such as patient interfaces. For example, the present technology relates to component designs suitable for acoustic identification of the component, such as acoustic identification of a respiratory therapy interface or mask.

### BACKGROUND OF THE TECHNOLOGY

### OSA and CPAP

Obstructive sleep apnoea (OSA) is a common form of sleep breathing disorder which affects an estimated 1 billion people globally [1]. It is characterised by a partial or complete closure of the upper respiratory tract during sleep, which causes hypopnea or apnoea [2]. This disrupted or disordered breathing directly causes hypoxia and hypercapnia, which causes increased respiratory efforts, sympathetic overactivity, oxidative stress and systemic inflammation [3]. OSA is associated with increased risk of cardiovascular problems, impaired cognitive function, excessive daytime sleepiness, and depression [4-7]. OSA is estimated to cost the Australian economy $13.1 billion per year, with major contributions being in lost productivity and healthcare [8]

The current gold standard treatment for patients with moderate to severe OSA is positive airway pressure (PAP) therapy, such as continuous positive airway pressure (CPAP) therapy, which provides positive pressure to the airways and prevents the airways from closing during sleep [9]. CPAP was invented by Colin Sullivan in 1981 as a method of mechanically preventing upper airway occlusion by providing a “pneumatic splint” [10]. During normal breathing the diaphragm contracts and moves downward to inhale and relaxes to exhale. In contrast, PAP forces the patient to exert effort during exhalation to oppose the incoming positive pressure [11].

Current PAP products require a device which generates pressure and passes air through the humidifier, through a tube and to the attached patient interface such as a mask. CPAP masks can be categorized as: nasal, oronasal, or full face, and nasal pillows. Within these categories exist many variations. They include variants with the tubing either connected on top of the head or at the nose, and various models from multiple manufacturers using different materials, venting mechanisms, and geometry [12].

### Mask Detection for Improved Patient Compliance

PAP is a highly effective treatment when used correctly, although patient compliance is a barrier to long term success [13]. Mask issues are commonly cited as the reason behind patient non-compliance [14]. Reasons for poor patient adherence include physical discomfort, social perception of PAP, and side effects such as skin irritation [15]. Therefore, efforts to improve the patient experience and remove barriers to long term adherence are crucial to improving the treatment of OSA. Remote assistance is an important component of modern PAP therapy, with studies showing that telemonitor care significantly improves PAP compliance [16]. CPAP devices can be cloud-connected and able to remotely provide a myriad of information to care providers.

Apparatus to deliver breathable gas to a patient typically includes a flow generator, an air delivery conduit, and a patient interface. A variety of different forms of patient interface may be used with a given flow generator. Furthermore, different forms of air delivery conduit may be used. In order to provide improved control of therapy delivered to the patient interface, it may be, for example, advantageous to measure or estimate treatment parameters such as pressure in the mask, and vent flow. In systems using estimation of treatment pressures, knowledge of exactly which mask is being used can improve therapy. For example, known flow generators include a menu system that allows the patient to select the type of peripheral components being used, e.g., by brand, method of delivery, etc. Once the components are selected by a clinician, the flow generator can select appropriate operating parameters of the flow generator that best coordinate with the selected components.

Similarly, the therapy device typically needs to be set to the correct flow setting depending on the mask model for optimal therapy, however current devices rely on the clinician or patient manually inputting the setting. As more masks are developed and released to market, the settings configurations are becoming increasingly unintuitive, and it is more likely that settings are set incorrectly. For example, ResMed devices have Full Face, Nasal or Pillows settings options, but some next generation pillows masks are best suited to the nasal setting. Patient compliance may even decrease when CPAP devices are on the incorrect setting.

Additionally, a therapy system may be coupled with a digital assistant, such as the ResMed My Air application, that can aid in trouble shooting therapy issues directly with the patient. Troubleshooting content, such as educational information about how best to use and adjust the mask, can be significantly more effective and tolerable, if it is provided for the specific therapy set-up and/or therapy issues. For example, if the system determines an issue with unintentional leak, providing information to the patient about how to adjust and wear the specific mask they are using can be significantly more effective than providing more generalised patient coaching.

The present technology may provide improvements to known apparatus to facilitate the coordination between the flow generator and the peripheral components based on acoustic detection to distinguish or identify particular components. In general, PAP devices, as well as other respiratory therapy devices, such as high flow therapy devices that use interfaces that do not generally form a seal with the patient, would benefit from an economical and effective method of automatically identifying which mask is connected to the system. As several manufacturers have a growing number of patient interfaces (e.g., masks) on the market, it is becoming increasingly important to implement a way to automatically collect data on patient interface models being used to provide better help to patients, administer the correct therapy settings, and inform future mask design.

There are no currently known CPAP devices on the market that fully and economically detect and identify the connectable masks; settings are typically set manually by the clinician or user. Various technologies are shown in FIG. 1. Ideally, a solution would be accurate, able to identify current and past masks from all suppliers, automatic, require no action by patients, and low cost.

Other proposals include incorporating electronics or sensors to the mask which communicate the mask model to the device. These options require no input from the user but do not resolve the issue of detecting competitor masks or legacy masks.

An acoustic detection method by ResMed was first developed by Liam Holley in 2009 [22, 29] for the identification of masks in a respiratory treatment system, as described in U.S. Pat. No. 10,773,038 B2 (hereinafter “Holley”), the entire disclosure of which is incorporated herein by reference. [29]. This may involve using a single microphone in a CPAP device to detect acoustic signals travelling along the tube waveguide, and characterising the connected mask based on the reflected sound. This method is low-cost due to requiring only one microphone, does not require user input, and has the potential to identify legacy and competitor masks.

### BRIEF SUMMARY OF THE TECHNOLOGY

The present technology is directed towards providing medical devices, or the components thereof, that may be used in the management, monitoring, amelioration, treatment, and/or prevention of respiratory related conditions having one or more of improved comfort, cost, efficacy, ease of use and manufacturability.

Some example configurations of the present technology relate to apparatus used in the management, monitoring, detection, diagnosis, amelioration, treatment or prevention of a lymphatic system disorder.

The technology described herein may allow for more accurate mask classification, such as by a classifier, (e.g., an SVM classifier or neural network). Patient interfaces may be provided with improved geometrical features to better affect automatic identification by acoustic detection, such as with an impulse response function and cepstrum determined by the system. Patient interfaces may be provided with improved material construction, such as by selective inclusion of certain material differences in portions of mask structures, to better affect automatic identification by acoustic detection. Such implementations may improve masks with low acoustic identification accuracy, improve the accuracy of the mask identification classifiers, improve feasibility of acoustic detection. Such implementations may develop new patient interface structures (such as withing the flow path) for producing unique cepstra.

Some example implementations of the present technology may include a patient interface model. The model of patient interface may be configured for delivering a respiratory therapy to a patient via an air circuit from a respiratory therapy device. The patient interface model may include an inlet that may be connectable with the air circuit. The patient interface model may include a plenum chamber integrated with the inlet and in fluid communication with the inlet. The inlet and plenum chamber may form a flow path for the respiratory therapy. The plenum chamber may be configured for coupling with a respiratory system of the patient for delivery of the respiratory therapy to the patient. The patient interface model may include a positioning and stabilising structure configured to engage the plenum chamber with the patient, such as with a seal forming structure or cushion. The patient interface may be adapted with one or more first model specific acoustical features in the flow path that are configured to produce an acoustic response to sound in the flow path that distinguishes the model of the patient interface from different patient interface models that have respectively different one or more model specific acoustical features.

In some implementations, the one or more first model specific acoustical features may include a difference in material composition of at least a portion of a wall section of the flow path. The difference in material composition may include a preceding section and an interceding section, where each may be formed of a different material composition to the other. A repeated sequence of the preceding section and the interceding section may form a plurality of interceding sections. The one or more first model specific acoustical features may include a count of the plurality of interceding sections that may be different from a count of a plurality of interceding sections of the different patient interface models that have respectively different one or more model specific acoustical features.

In some implementations, the difference in material composition may include a first material composition that is different from a second material composition of the different patient interface models that have respectively different one or more model specific acoustical features.

In some implementations, the one or more first model specific acoustical features may include a length of a portion of the flow path that may be different from lengths of the portion of the flow path of the different patient interface models that have respectively different one or more model specific acoustical features. The portion of the flow path may include a sounding chamber. The sounding chamber may be an expansion of the flow path, which may be a change in diameter of the flow path. The length of the portion of the flow path that is different from lengths of the portion of the flow path of the different patient interface models may be different by at least five millimeters. In some implementations, each length of the lengths of the portion of the flow path of the different patient interface models are different from each other by at least five millimeters.

In some implementations, the one or more first model specific acoustical features may include a first deviation or gradient in a portion of the flow path. The first deviation or gradient may comprise a change in diameter in the portion of the flow path. The one or more first model specific acoustical features may include a second deviation or gradient in the portion of the flow path. The second deviation or gradient may comprise a change in diameter in the portion of the flow path. The first deviation or gradient and the second deviation or gradient may form a sounding chamber having a first length that may be different from lengths of sounding chambers in flow paths of the different patient interface models that have respectively different one or more model specific acoustical features. The flow path of the patient interface may include additional deviations or gradients that form a further sounding chamber having a second length that may be different from lengths of further sounding chambers in flow paths of the different patient interface models that have respectively different one or more model specific acoustical features. The first length may be different from the second length by at least five millimeters.

In some implementations, the one or more first model specific acoustical features may include a difference in material thickness of at least a portion of a wall section of the flow path. The difference in material thickness may include a preceding section and an interceding section, each formed of a different material thickness to the other. The flow path of the patient interface may include a repeated sequence of the preceding section and the interceding section that form a plurality of interceding sections. The one or more first model specific acoustical features may include a count of the plurality of interceding sections that may be different from a count of a plurality of interceding sections of the different patient interface models that have respectively different one or more model specific acoustical features. The difference in material composition may include a first material thickness that may be different from a second material thickness of the different patient interface models that have respectively different one or more model specific acoustical features.

In some implementations, the one or more first model specific acoustical features in the flow path may be formed in the inlet. The inlet may include a swivel. In any of these implementations, the inner surface of the air circuit from the respiratory therapy device may be substantially smooth as in more described herein.

Various aspects of the described example configurations may be combined with aspects of certain other example configurations to realize yet further example configurations. It is to be understood that one or more features of any one example may be combinable with one or more features of the other examples. In addition, any single feature or combination of features in any example, or a combination of features from two or more examples, may constitute patentable subject matter.

Other features of the technology will be apparent from consideration of the information contained in the following detailed description.

The subject headings used in the detailed description are included only for the ease of reference of the reader and should not be used to limit the subject matter found throughout the disclosure or the claims. The subject headings should not be used in construing the scope of the claims or the claim limitations.

### BRIEF DESCRIPTION OF DRAWINGS

FIG. 1 illustrates technologies for mask identification.

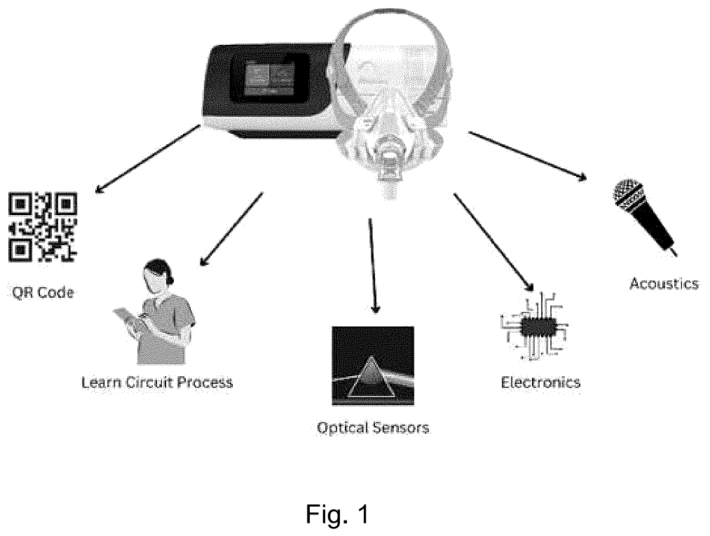

FIG. 2 illustrates an impulse response function (IRF) of a system showing reflection of a mask at 2L/c and a humidifier at 2x/c).

FIG. 3 illustrates IRF changes in response to changes in component cross-section.

FIG. 4A illustrates an example system including an integrated part, that may serve as a model for a mask or an acoustic structure or sounding chamber for identifying a mask when integrated with a mask, in accordance with some aspects of the present disclosure.

FIG. 4B illustrates an example cepstrum corresponding to such an integrated part in accordance with aspects of the disclosure.

FIG. 5 illustrates an example of such an integrated part in accordance with aspects of the disclosure.

FIG. 6 illustrates an example integrated part operatively connected to an air circuit in accordance with aspects of the disclosure.

FIG. 7 illustrates an example integrated part operatively connected to an air circuit in accordance with aspects of the disclosure.

FIG. 8A shows a system including a patient 1000 wearing a patient interface 3000, in the form of nasal pillows, receiving a supply of air at positive pressure from a respiratory pressure treatment (RPT) device 4000. Air from the RPT device 4000 is humidified in a humidifier 5000, and passes along an air circuit 4170 to the patient 1000. A bed partner 1100 is also shown.

FIG. 8B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 8C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000.

FIG. 9 shows a patient interface in the form of a nasal mask in accordance with one form of the present technology.

FIG. 10A shows an RPT device in accordance with one form of the present technology.

FIG. 10B is a schematic diagram of the pneumatic path of an RPT device in accordance with one form of the present technology. The directions of upstream and downstream are indicated.

FIG. 10C is a schematic diagram of the electrical components of an RPT device in accordance with one form of the present technology.

FIG. 10D is a schematic diagram of the algorithms implemented in an RPT device in accordance with one form of the present technology.

FIG. 10E is a flow chart illustrating a method carried out by the therapy engine module of FIG. 10D in accordance with one form of the present technology.

FIG. 11 shows an isometric view of a humidifier in accordance with one form of the present technology.

### DETAILED DESCRIPTION

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### Acoustic Detection

### Acoustic Applications

Sound waves are longitudinal waves created by particle vibrations in a medium. The The vibrations create compressions and rarefactions, or a repeating pattern of high and low pressure regions [30]. When a microphone measures sound it typically measures the pressure fluctuation, just as the human ear does [31]. Sound waves are characterized by their wavelength, amplitude, and frequency. Humans can hear sounds of frequencies between 20 Hz and 20000 Hz [32]. Waves with a frequency below 20 Hz are infrasonic while waves with a frequency above 20000 Hz are ultrasonic [33]. Microphones can sense sound waves beyond the frequency range of human hearing. Acoustics as a field has a variety of applications including improving pleasant sounds such as music, minimising unpleasant sounds such as machinery, and using acoustic information to deduce further information about an object or system.

### Cepstral Analysis

Cepstral analysis may be used to extract the impulse response function (IRF) and identify a therapy component coupled to a respiratory therapy delivery tube, such as for identifying the mask attached to a CPAP system [29]. This method relies on component cepstra to be sufficiently different so the cepstra acts as the mask signature and the mask model can be identified by a ML classifier. As yet, the literature has not explored how more subtle changes in an airpath seen in CPAP masks affect the cepstrum. The use and application of cepstra is described in U.S. Pat. No. 10,773,038, the entire disclosure of which is incorporated herein by reference.

### Acoustic Methodologies

Acoustic impedance is a property of a medium or object is a measure of acoustic flow due to acoustic pressure [89]. It denotes the amount of sound pressure generated by the vibration of molecules at a particular frequency in a specific acoustic medium. The acoustic impedance of a material depends on its density and the velocity of the soundwave through the material medium, while in a duct the acoustic impedance also depends on the cross-sectional area and the wall stiffness. Many methods are well researched for measuring acoustic impedance of materials including in ducts [90, 91].

The cepstral analysis method proposed in Holley has key advantages in the context of CPAP when compared with other acoustic impedance measurement methods [22, 29]. For example, a single microphone may be offset from the airpath, fixed in a single position, and covered with a silicone membrane. This is possible due to this method being more robust to variations in geometry of the coupling, or drift in the microphone response as distortions are applied to both the incident and reflected sound waves and are removed during signal processing.

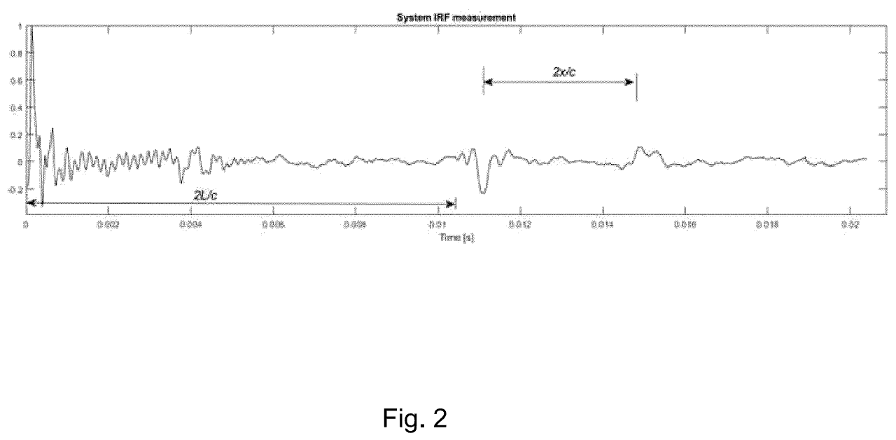

The IRF is defined as the system output in response to an impulse in pressure. The impulse must be sufficiently short so that the magnitude of its spectrum is flat across frequencies of interest. Sound is produced by the device, such as a flow generator, travels through the tube and to the mask where it is reflected and travels back [22]. The signal received by the microphone is the sound from the device, and after some delay is the sound reflected and filtered by the tube and mask. The sound travels down the length of the system and back again. The delay is the distance travelled divided by the speed of the sound:

$$t = \frac{2 L}{c}$$

Where/, is the distance, and c is the speed of sound. For example, FIG. 2 shows the IRF of a CPAP system with a mask connected. After a time of

$$\frac{2 L}{c}$$

the signature associated with the mask is seen. After

$$\frac{2 x}{c}$$

seconds the signature associated with the humidifier is seen as the sound has returned to the humidifier and reflected to the microphone again.

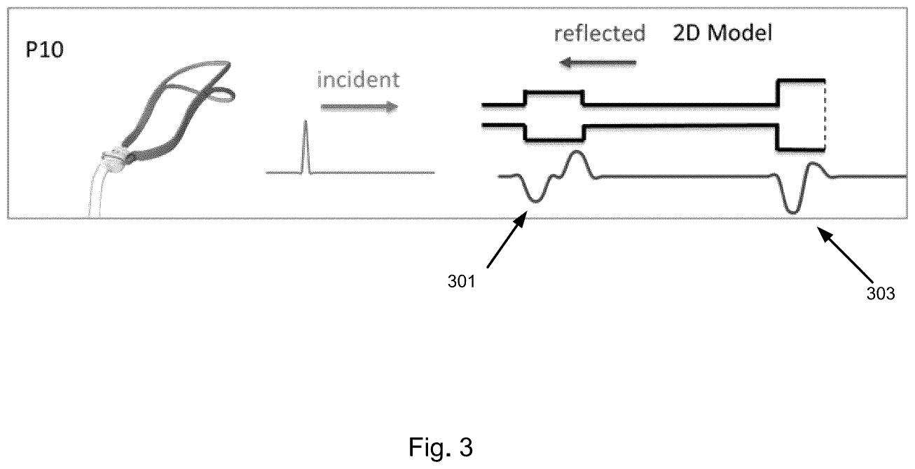

Some energy is reflected where there is a disturbance in the cross-section. It appears as a negative peak for an expansion, and a positive peak for a contraction, as shown in the simplified schematic of a ResMed P10 mask in FIG. 3. This means the cepstrum essentially maps the change in pressure against time which can be converted to distance if the speed of sound is known. Therefore, the cepstrum effectively shows the pressure changes along the PAP system from the device, through the tube, through the mask and to the patient wearing the mask. Each mask model has different geometry and materials along its cross-section which has been seen in their cspstra during both bench tests and therapy. Therefore, provided each mask's cepstrum is sufficiently unique, a machine learning model such as an SVM classifier can be trained to identify the mask coupled to a system at a given time.

### Fundamental Frequency and Harmonics

The fundamental frequency, often referred to as the first harmonic, is the lowest frequency of a periodic waveform. Whether tuning the resonant frequencies of a musical instrument or analysing the flow of air in industrial pipes, the fundamental frequency is the governing principle at its basis [99]. This phenomenon plays a pivotal role in diverse fields, from the design of wind instruments to the optimization of ventilation systems [100, 101].

The fundamental frequency (f₀) of a pipe is a function of the pipe's length (L) and the speed of sound (c) which depends on the medium it travels through [102]. A pipe is classified based on whether both ends are closed, both ends are open, or one is closed and one is open. For a closed-closed pipe, the fundamental frequency is found by:

$${f}_{0} = \frac{c}{2 L}$$

For an open-closed pipe, the fundamental frequency is found by:

$${f}_{0} = \frac{c}{4 L}$$

### Acoustics in CPAP

A CPAP system has distinct characteristics that affect its acoustic behaviour. All audible sound is minimised by design to reduce its disturbance on the sleeping patient. However, sound remains present in the system as pressure moves through the system to the patient [103]. The sound from the CPAP system includes both random and periodic elements, shown as discrete peaks within its broadband frequency [104].

CPAP devices are generally rotodynamic pumps which generate pressure via a rotating impeller that accelerates air around its axis [105]. The air velocity at the impeller tip is often over ten times faster than the air at other parts of the system and is therefore a primary location for turbulence. Turbulence is random in nature, and therefore the impeller is the primary source of random noise [104]. Other less significant sources of turbulent energy are at sharp corners such as in the tube during use, in the humidifier, or points with restricted cross-sectional area [106].

Periodic pressure fluctuations are caused due to imbalances or misalignments in the rotating shaft, electromagnetic force fluctuations from the motor coils being periodically energised and the rotating impeller blade tips causing periodic pressure pulses at the blade passing frequency [104]. These pressure fluctuations produce periodic noise which can produce harmonics of their fundamental frequency at a multiple of the shaft speed [107].

CPAP tubes are corrugated to increase the strength and bendability, like vacuum tubes. Corrugated tubes or pipes can emit whistling sounds during air flow not observed in smooth pipes of similar geometry [108]. Vibrations in a corrugated pipe are primarily caused by vortex shedding around cavities, which causes the acoustic field and aerodynamic field to interact [109]. Various models have been proposed to understand acoustics in corrugated pipes, including the Cummings model [110], singing tube model [111], and Binnie model [112]. CPAP tubes are designed with noise as a key consideration due to being used during sleep, so noise is minimised, and tonal sounds are chosen by design to be less disruptive to sleep.

The tube acts as a waveguide to direct the pressure to the patient. The CPAP system is a class II medical device that delivers pressure to a patient, directly interfacing with the patient's skin and breath [113]. Installing a bare microphone, or multiple microphones, within the airpath is undesirable in the PAP system. For example, the airpath can be at approximately 80% relative humidity during some forms of therapy due to use of a humidifier and exhaled breath from the patient which can corrode the electronics [114]. Materials in the airpath must also be biocompatible and approved for patient safety [113]. A single microphone method is more robust to offsetting the microphone from the airpath and adding silicone membrane which protects the microphone from humidity and the patient from the electronics [29]. The microphone would be integrated in the CPAP device which has a design life of three to five years compared to the mask or tube which typically last less than a year [12]. This minimises the hardware required which reduces costs.

Factors that Affect the Cepstrum of a CPAP system

Geometry and materials are the major contributors to the acoustic impedance and acoustic signature of components of a CPAP system. Expected changes in geometry arise from various configurations of CPAP products including the manufacturer and model of the device, tube, and mask. Within each model exists variance caused by manufacturing tolerances, or the system layout, although these are relatively minor. Tube length manufacturing tolerances are typically within 1 cm. Compressing or stretching the tube axially during use deforms the tube elastically for small forces and plastically for larger forces, although the tube will typically tear before significant stretching is seen. The tube may be squished laterally during use which reduces its diameter and increases pressure.

In fluid mechanics the Hagen-Poiseuille and Darcy-Weisbach equations [115, 116] dictates that the pressure drop across a long cylindrical pipe during laminar flow is

$$Δ P = \frac{8 μ L Q}{π {R}^{4}} = \frac{128 μ L Q}{π {D}^{4}}$$

While during turbulent flow the diameter has an even more significant effect. Diameter is taken as approximately the fifth power.

The pressure drop is proportional to length but is inversely proportional to the diameter at a higher power. This means diameter or cross-sectional area has far more impact than length when considering fluid dynamics and pressure losses through the system. However, variations in length are important for cepstral analysis as it directly increases the time taken for pressure transient to travel and therefore shifts peaks in the cepstrum along the x-axis [83].

The speed of sound varies in the CPAP system depending on the conditions of use. The acoustic signature depends on the speed of sound which is affected by temperature and the medium through which it's travelling. Heated tubes maintain a constant temperature throughout the night depending on the settings, typically between 15 to 30° C. but unheated tubes may be at a greater range of temperatures [117]. Even still, this range would remain within liveable temperatures for humans and therefore will not contribute significantly to speed of sound variance. Humidity in the airpath negligibly increases the speed of sound. The air composition along the airpath varies as CO₂ is exhaled periodically by the patient. Increased CO₂ reduces the speed of sound [118]. CPAP systems are designed to avoid accumulation of CO₂ for patient safety.

Applying acoustic detection to determine the mask in use is unique in that the object being identified can have its IRF or acoustic impedance manipulated to improve the effectiveness of a characteriser algorithm or classifier (e.g., a machine learning classifier). Specifically, as discussed in more detail herein, unique structural geometries and/or materials may be integrated into the patient interface's flow path to make the patient interface more unique from the acoustic detection perspective and therefore easier for an algorithm, such as the cepstrum based classifier, to identify. Moreover, with mask detection there is a discrete, correct answer with finite options since it concerns a detection with a known collection of different mask types/models. This has a key advantage: the algorithm does not need to determine the exact acoustic impedance. To this end, the masks structures described herein can be identified because they are designed so that their acoustic signatures are sufficiently unique.

For example, typical respiratory patient interface (e.g., mask) structures may be modified to include one or more acoustical features that do not generally provide any therapeutic benefit to the interface and otherwise do not negatively affect therapy provided with the patient interface but the acoustical feature(s) provide differences in structure that can produce, when acoustically sensed as previously described, a unique acoustic signature for identification of a particular model mask and thereby differentiate it from other model masks. The acoustical feature(s) may determine the cepstrum, or acoustic signature, of the system. The added acoustical feature(s) may be generally similar across different models of patient interface but have detectable differences between the models. However, the added acoustical feature(s) of a particular model are typically substantially the same for all masks of that particular model. Such acoustical feature(s) (AF) may, for example, be a change in material composition of a section or part of a flow path of the patient interface, a change in a dimension, such as a length, of a section or part of a flow path of the patient interface, and/or an expansion and/or contraction of a section (e.g., diameter) or part of a flow path of patient interface. While any one of such changes may be implemented to produce a sufficiently different acoustic signature between different models of patient interface, any combinations of such changes may be implemented to improve uniqueness of the acoustic signatures between different models.

For example, in some implementations, an integrated inlet tube and/or connection port to the mask plenum chamber that is present on many masks could be lengthened or shortened slightly so as to provide different lengths for different models. Such a difference in length may be, for example, greater than five-millimetres. Such changes may then provide differences in the acoustic response according to the different lengths and thereby provide a basis to acoustically identify such different models, such as with a classifier trained to distinguish their IRFs.

Similarly, in some implementations, an integrated part of a patient interface, such as an inlet (e.g., a connection port, such as in an integrated swivel) to the plenum chamber of a mask, may include one or more deviations and/or gradients in a wall of the breathable gas flow path through the inlet. The one or more deviations change (e.g., increase(s) and/or decrease(s)) a cross-sectional area of a section of the flow path. Such a change in the flow path may create a sounding chamber or a plurality of such sounding chambers. An example of such an acoustical feature (AF) is shown in FIG. 3. For example, a deviation or gradient may be a step change in the surface of the wall or a gradual slope resulting in the beginning of a sounding chamber. (See, e.g., left side 301 of AF in FIG. 3.) The sounding chamber may be further bounded by an end (see, e.g., right side 303 of AF in FIG. 3), such as with additional deviation(s) or gradient(s) of the wall in structure of the flow path that may return the flow path to its original cross-sectional area. The structure(s) of the sounding chamber(s) may be substantially same for all patient interfaces of a particular model. However, one or more differences (e.g., dimension(s)) in the structure of the sounding chamber may be introduced for different models of patient interface, to permit the structures of the sounding chamber to serve as different and unique acoustic identifiers for each of the particular models. For example, different models of the patient interface may have differences in the depth of the deviations/gradients of the chamber. Similarly, different models of the patient interface may have differences in the length of the sounding chamber (e.g., the distance between a beginning deviation/gradient and an end deviation/gradient.) In this way, different models of patient interface may be manufactured so as to include detectable acoustic distinctions such as for different mask models that might not otherwise be distinguishable by acoustic detection.

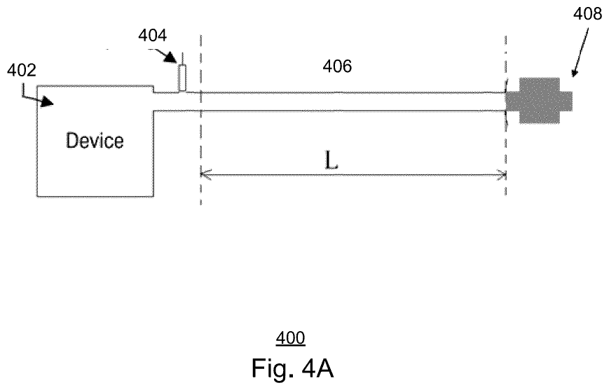

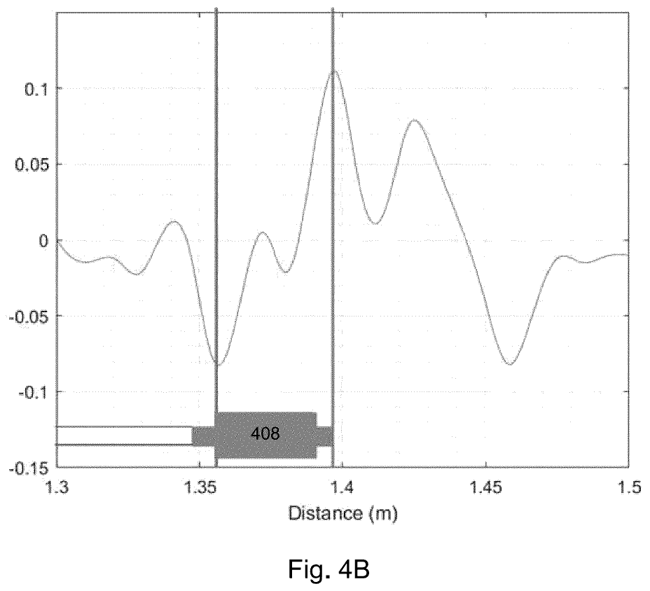

FIG. 4A illustrates an example system 400 including a device 402, one or more acoustic sensors 404, an air circuit 406, and an integrated part 408. Device 402 may be any respiratory pressure therapy (RPT) device configured to supply a flow of pressurised air to the patient via air circuit 406 having a length L. The one or more acoustic sensors 404 may be any sensors configured to detect acoustic signals traveling along the air circuit 406. In some instances, the one or more acoustic sensors 404 may include a microphone. Acoustic signals detected by the one or more acoustic sensors 404 may be used to generate an impulse response function and cepstrum. FIG. 4B illustrates an example cepstrum where an example acoustic signature of the integrated part 408 may be seen. While illustrated separately in FIG. 4A, in some instances, the one or more acoustic sensors 404 may be included in the device 402.

The integrated part 408 may be a portion of the patient interface such that it is positioned at an end of the air circuit 406 opposite the device 402. In some instances, the integrated part 408 may be a component separate from the patient interface and may be positioned at an end of the air circuit 406 opposite the device 402. In some instances, the integrated part 408 may be positioned on the device 402 side of the air circuit 406 or at any other point along the air circuit 406.

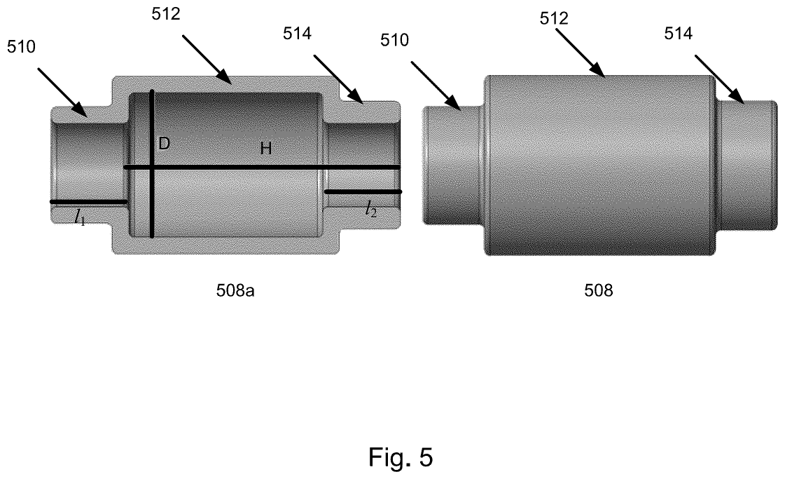

FIG. 5 illustrates a cross-section view 508a of an example integrated part 508, which may serve as an example sounding chamber for purposes of the patient interface identification technology described herein, when integrated with a patient interface. Integrated part 508 includes an inlet region 510, a middle region 512, and an outlet region 514. The cross-section view 508a illustrates that integrated part 508 includes a diameter D, a length H, an inlet length/1, and an outlet length/2. The diameter D is the diameter of the middle region 512, the length H is the length of the middle region 512 and the outlet region 514, the inlet length/1 is the length of the inlet region 510, and the outlet length/2 is the length of the outlet region 514. Each of these measurements of the integrated part 508 may be varied to produce unique acoustic signatures. While illustrated as constant in FIG. 5, the diameter D may be variable along the length of the middle region 512. Additionally, or alternatively, an integrated part, such as integrated part 508, may include a plurality of middle regions such as a first middle region, a second middle region, etc. Each of the plurality of middle regions may be dimensioned differently or the same. In some instances, the plurality of middle regions may be separated by one or more transition regions of different dimension, material, or both. In some instances, the length H may be differed by at least 5 mm between different models to produce a unique acoustic signature. In some instances, the length H may be differed between different models so that each differs by 5 mm from a first model (e.g., 5 mm difference for a second model, an additional 5 mm (i.e., 2 times 5 mm) for a third model, and another 5 mm (i.e., 3 times 5 mm) for a fourth model etc. In some instances the length H may be differed by at least a length difference in a range of between 5 mm and 10 mm, or more e.g., for different models to produce a unique acoustic signature.

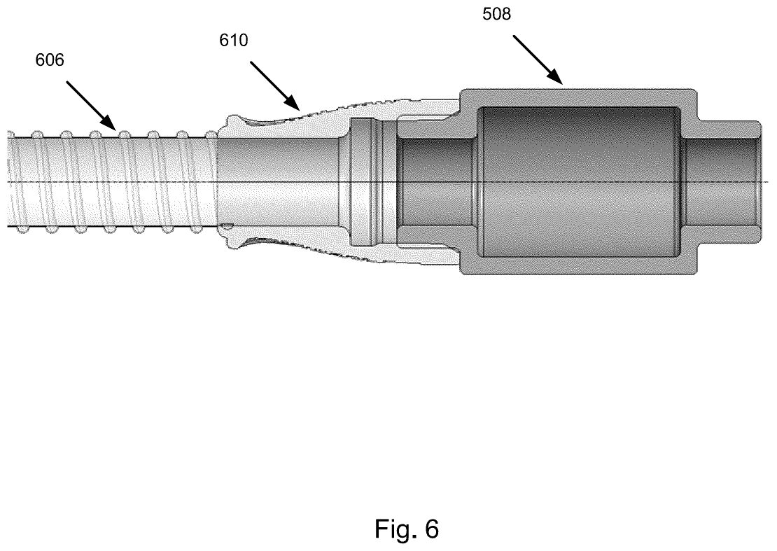

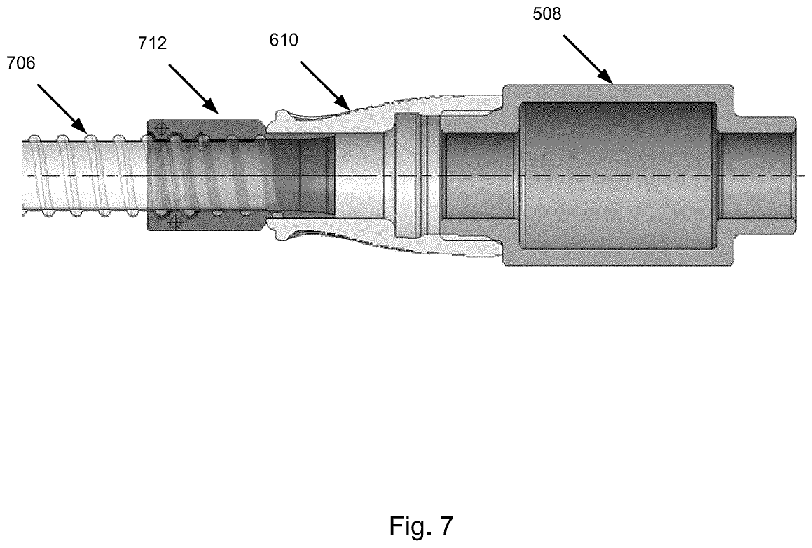

FIG. 6 illustrates integrated part 508 connected to an example air circuit 606 via a cuff 610. In some instances, an extra component, such as an additional cuff may be included to couple an integrated part to an air circuit. For example, FIG. 7 illustrates integrated part 508 connected to an example air circuit 706 via cuff 610 and additional cuff 712. In this regard, the integrated part may be compatible with various air circuits corresponding to various devices.

Similarly, in some implementations, a mask may be designed using a number of components (which may include but is not limited to an integrated component as discussed above) as in an assembly where several components provide any of the acoustical feature(s) described herein. The mask may then be assembled such that when a number of such acoustically enabled components are assembled (for example, in the manufacturing process), they introduce a unique arrangement of the acoustical feature(s) (of the arranged components) to the patient interface, and as such, a unique acoustic response signature is introduced into the patient interface. Moreover, variation in the assembled order may alter the reflective Impulse Response Function (IRF) of the assembled mask system, such as by altering the distance of various acoustic features (AF) from a reference point (such as a sensor in the device or Flow Generator). In this way, by varying the order of assembly of some sub-components, such as where the components each have different ones of the acoustical feature(s). In this way, providing arrangeable acoustically detectable components, it is possible to greatly increase the number of unique acoustic identifiers, without necessarily increasing the number of manufacturing tools required. Moreover, additional unique acoustic identifiers can be produced by increasing or decreasing the number of such acoustically enabled components that are applied to the assembly.

Additionally, or alternatively, changes in material composition of the patient interface, such as in a wall of a flow path of an inlet to a mask and/or an integrated component as discussed above, may be similarly implemented to produce detectable model-based acoustic differences. For example, one or more discrete interceding section(s) (e.g., one or more ring sections of a round conduit) of a wall of a flow path, such as of an inlet to a plenum chamber of the mask, may be made of material that is different from a material of a preceding and/or succeeding section of the wall of the flow path. The interceding section, due to the difference in material composition, may then serve as an acoustic feature when the material has a different acoustic response compared to the acoustic response of a material of a preceding and/or succeeding section. For example, the interceding section, such as when forming a part of an acoustic chamber as described herein or a more constant cross-sectional portion of a flow path, may provide a more rigid material portion, or a more flexible material portion, to change the acoustic reflection character of the structure in the interceding section. Such a difference may then provide a detectable acoustic response, such as with the aforementioned acoustic detection methods. Moreover, additional variations in the configuration of such material composition difference section(s), such as length of the section, spacing between sections, and/or number of interceding sections, may be applied to different models to provide detectable model specific acoustic responses. For example, one model may be identifiable with two interceding sections and another model may be identified with four interceding sections. Similarly, one model of patient interface may be acoustically identifiable from one or more relatively shorter interceding section(s) and another different patient interface model may be acoustically identifiable by one or more relatively longer interceding section(s).

Similarly, changes in material quantity (e.g., thickness) of the patient interface, such as in a wall of a flow path of an inlet to a mask and/or an integrated component as discussed above, may be similarly implemented to produce detectable acoustic differences between different patient interface models. For example, one or more discrete interceding section(s) (e.g., one or more ring sections of a round conduit) of a wall of a flow path, such as of an inlet to a plenum chamber of the mask, may be formed with more (or less) material (e.g., thicker or less thick) compared to the material of a preceding and/or succeeding sections of the wall of the flow path. The interceding section, due to the difference in material thickness, may then serve as an acoustic feature when the material has a different acoustic response compared to the acoustic response of a material of a preceding and/or succeeding section. For example, the interceding section, such as when forming a part of an acoustic chamber as described herein or a more constant cross-sectional portion of a flow path, may provide a thicker material portion to change the acoustic reflection character of the structure in the interceding section. Such a difference may then provide a detectable acoustic response, such as with the aforementioned acoustic detection methods. Moreover, additional variations in the configuration of such thickness difference section(s), such as length of the section, spacing between sections, and/or number of interceding sections, may be applied to different models to provide detectable model specific acoustic responses. For example, one model may be identifiable with two interceding thicker (or thinner) sections and another model may be identified with four interceding thicker (or thinner) sections. Similarly, one model of patient interface may be acoustically identifiable from one or more relatively shorter interceding section(s) and another different patient interface model may be acoustically identifiable by one or more relatively longer interceding section(s).

Additionally, or alternatively, changes to air circuit may be included in the system to allow differing acoustic signatures to be more visible. For example, the internal surface of the air circuit may be substantially smooth, which as understood herein means that it will include minimal or no corrugations so as to reduce sound energy loss that diminish a generated cepstrum that is attributable to a patient interface at an end of the air circuit. Additionally, or alternatively, the air circuit may include minimal or no internal corrugations while still including external corrugations. In this regard, an inner surface of an air circuit may be relatively or fully smooth (i.e., no corrugations) while an outer surface of the air circuit may include corrugations. In another example, a length (e.g., length L illustrated in FIG. 4A) may be selected to support a particular fundamental frequency and allow for the production of a plurality of harmonics with large amplitude.

The current technology as described herein for providing one or more structural differences in certain points in a patient interface will be a simple way to make acoustic detection, such as with a cepstrum, more identifiable without having significant unwanted effects on therapy such as pressure losses, cost, or biocompatibility. Moreover, since changes in cross-sectional area (e.g., diameter) may have a greater impact on flow through the system when compared to mere changes to length of a part or section, maintaining consistent diameters may, with some implementations, permit system parameters to remain the same. However, minor differences with small changes may be sufficiently tolerable to permit both detection and avoid significant system performance changes.

### Some Benefits of Some Examples of the Present Technology

Implementations of the present technology provide improved patient interfaces for automatic identification in respiratory therapy systems, such as Positive Airway Pressure (PAP) devices or high flow therapy devices. For example, as current CPAP devices are cloud/network connected, the system information, including for example, mask identification, can be automatically sent to customer assistants to provide efficient and relevant support to patients. Current remote customer service via telephone or online requires the patient correctly identifying their mask model to the customer assistant which has proven inefficient and unreliable. Relying on self-reported data has been historically unreliable, with studies revealing that on average patients self-report CPAP use one hour per night longer than actual use [12]. Minimising time spent gathering information on the system allows more time spent providing help and advice to patients which benefits both the patients and customer assistants.

CPAP manufacturers often use data collected by CPAP devices and stored in the cloud to better understand trends in patient adherence, sleep, and number of apnoea events. With CPAP devices blind to the mask model in use, this data is missing key information that can be used to inform next generation mask designs to improve the patient experience.

Classifiers, such as support vector machine (SVM) classifiers, can more accurately identify and classify mask models based on their cepstrum, not only where mask designs are notably different such as a full-face mask compared to a nasal mask but also where similar designs have obscured acoustic identification.

Current methods of manually choosing the setting which determines the flow curve is flawed. An effective method of automatically communicating the connected mask to the flow generator requiring no user input will provide benefits to users, clinicians and system manufacturers.

### Optional Example Treatment Systems

An example embodiment of the system for automatic identification of a patient interface is discussed in more detail in the following sections. The below example is compatible with and may include one or more of the AF discussed above.

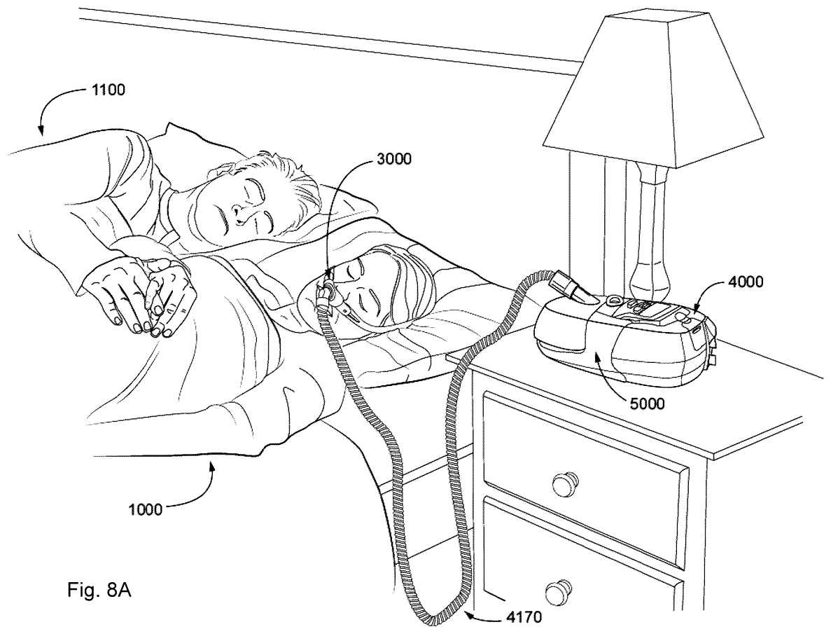

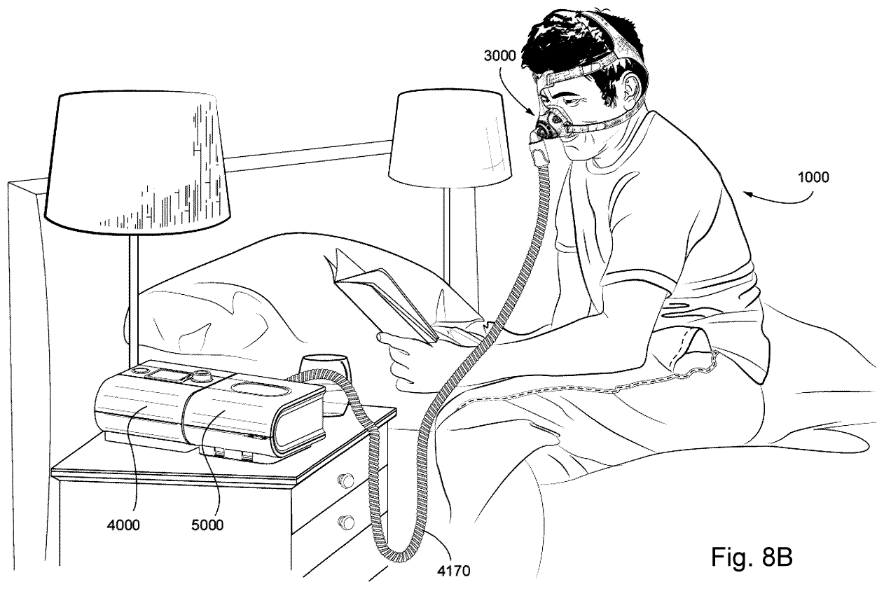

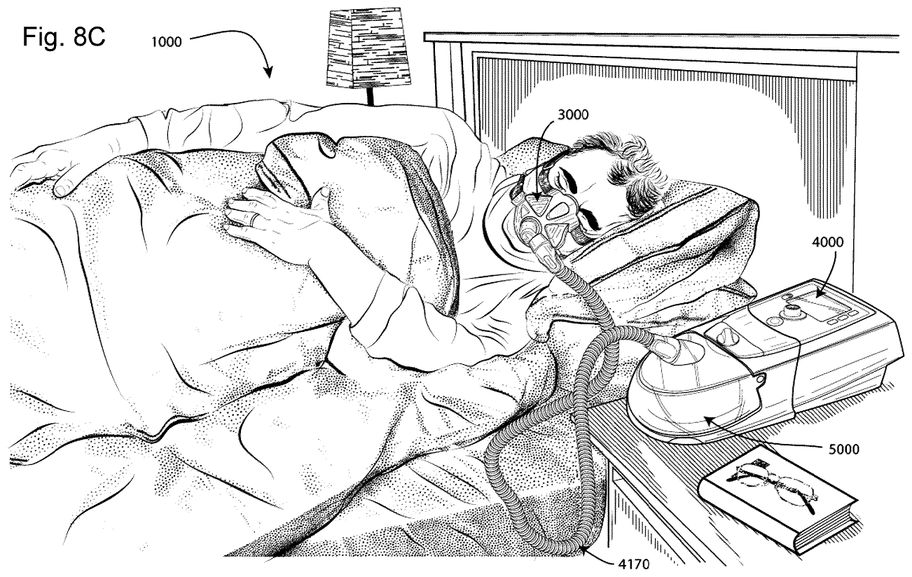

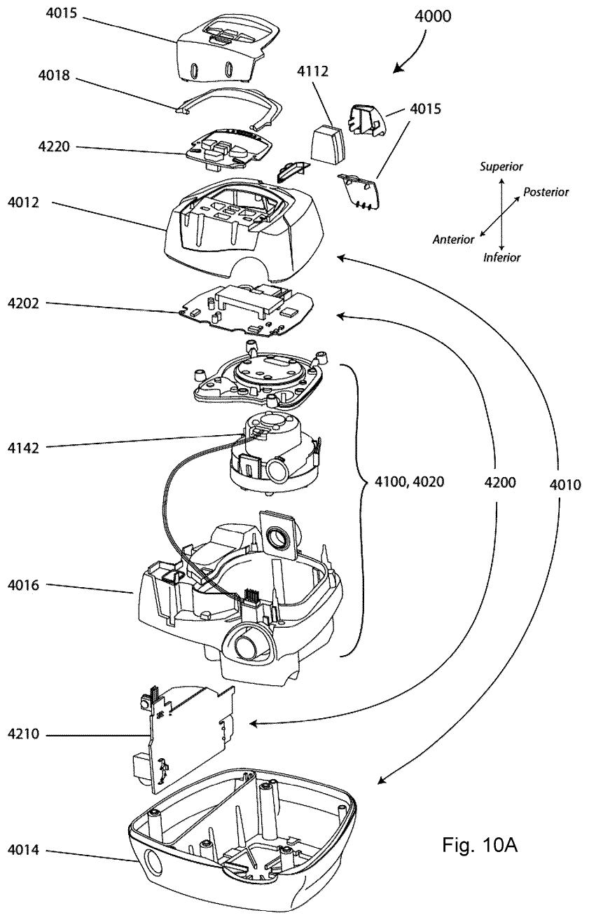

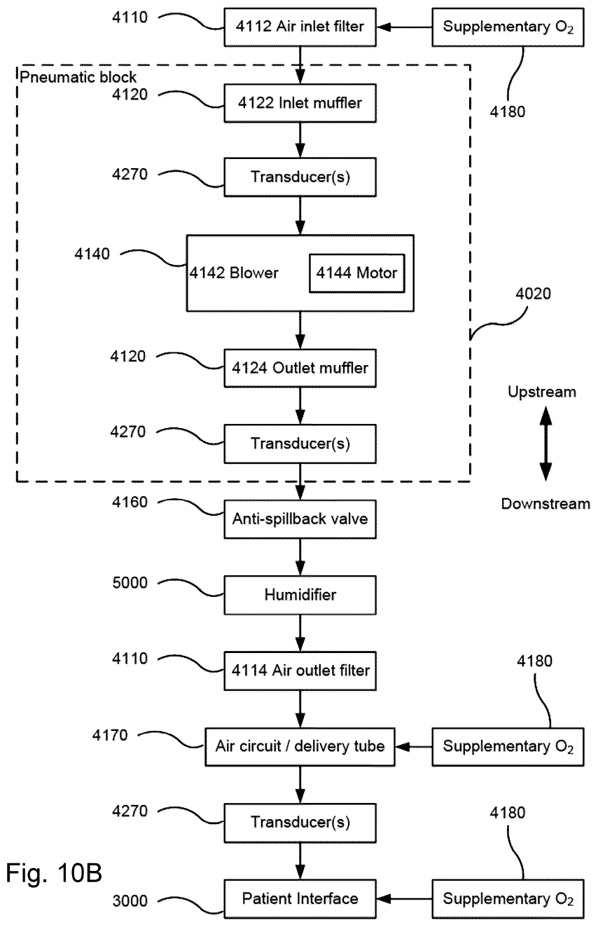

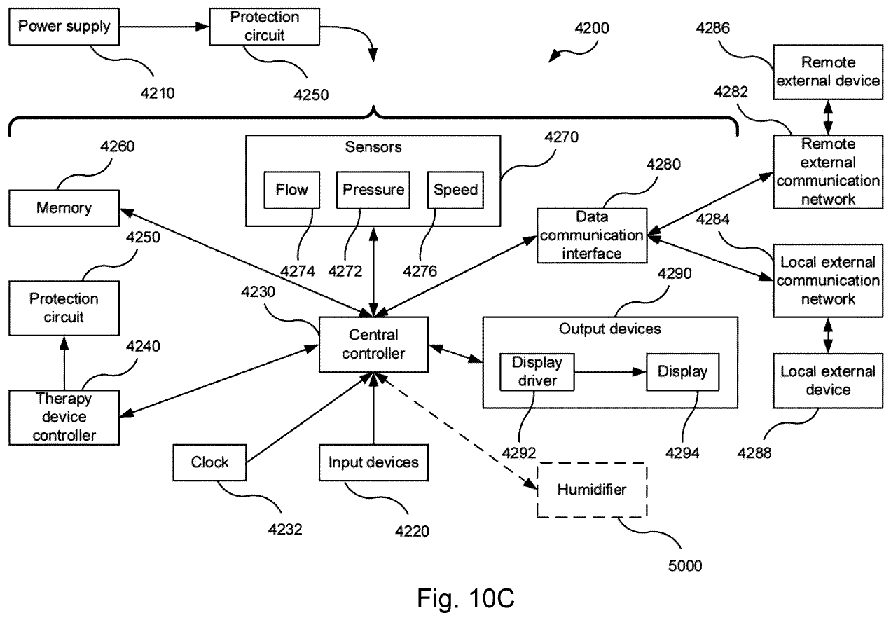

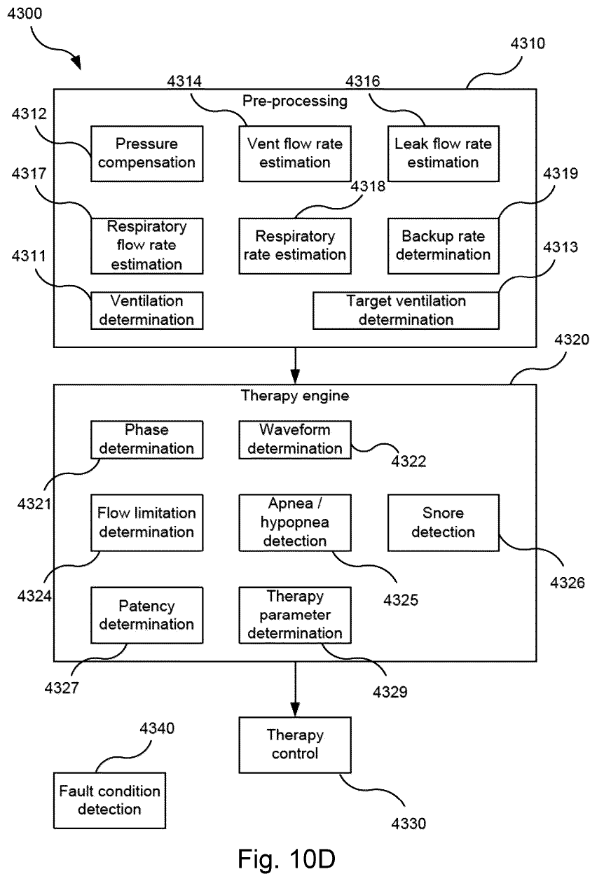

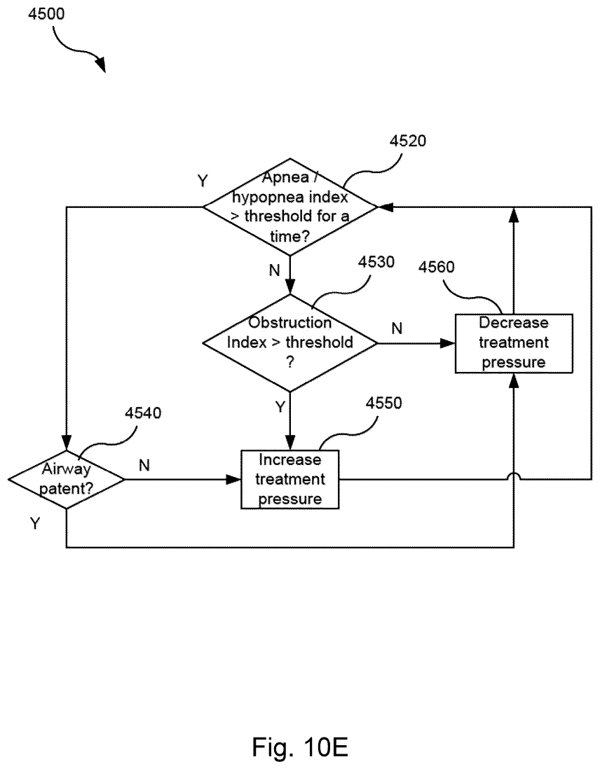

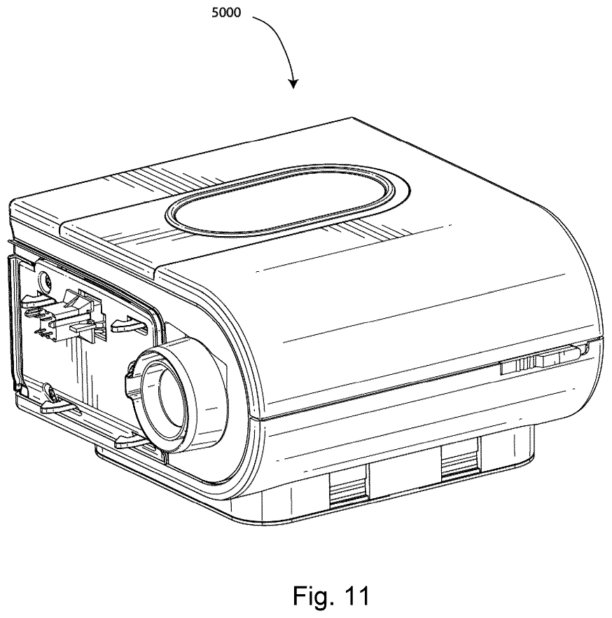

In one form, the system may treat and/or monitor a respiratory disorder. The system may be a respiratory therapy (RT) device such as an RPT device 4000 for supplying a flow of pressurised air to the patient 1000 via an air circuit 4170 leading to a patient interface 3000. The flow of air may be pressure-controlled (for respiratory pressure therapies) or flow-controlled (for flow therapies such as high flow therapy HFT). Thus, RPT devices may also be configured to act as flow therapy devices, such as when using a patient interface that does not use a seal that seals with the patient's respiratory system. In the following description, the RT or RPT device may be considered in reference to FIGS. 8A-11. The controller of the RT or RPT may be configured to perform the aforementioned acoustic detection methods, such as with one or more programmed processors and a microphone. The one or more processors, as discussed herein, may be included in the controller and/or be in a server such as on a network and in communication with the controller.

### 4.2 Patient Interface

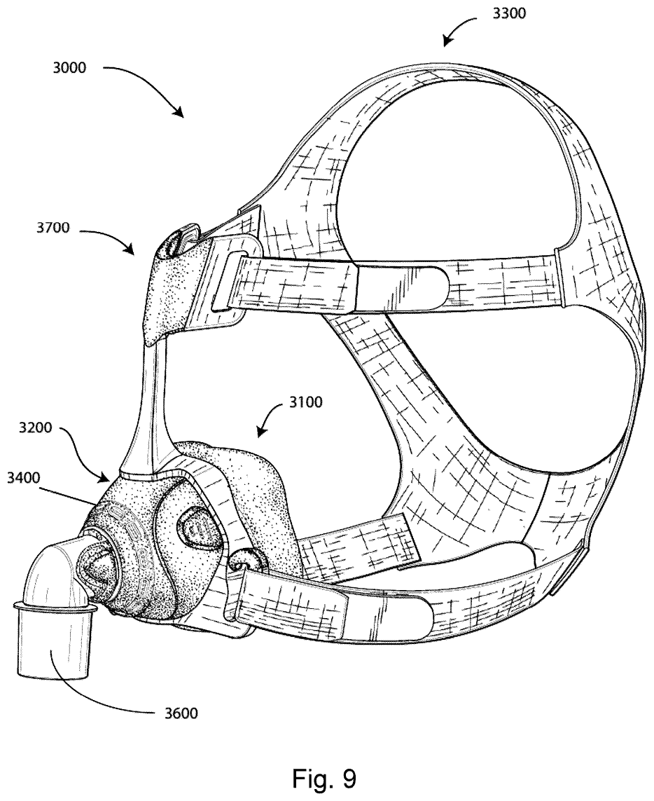

As shown in FIG. 9, a non-invasive patient interface 3000 in accordance with one aspect of the present technology may optionally comprise any of the following functional aspects: a seal-forming structure 3100, a plenum chamber 3200, a positioning and stabilising structure 3300, a vent 3400, a connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to an airway of the patient so as to facilitate the supply of pressurised air to the airway.

### 4.3 RPT Device

An RPT device 4000 in accordance with one aspect of the present technology comprises mechanical and pneumatic components 4100, electrical components 4200 and is programmed to execute one or more algorithms 4300. The RPT device 4000 may have an external housing 4010 formed in two parts, an upper portion 4012 and a lower portion 4014. In one form, the external housing 4010 may include one or more panel(s) 4015. The RPT device 4000 may comprise a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying pressurised air (e.g., a blower 4142), an outlet muffler 4124, and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 4.3.1 RPT Device Mechanical & Pneumatic Components

An RPT device 4000 may comprise one or more of the following components in an integral unit. In an alternative form, one or more of the following components may be located as respective separate units.

### 4.3.1.1 Air Filter(s)

An RPT device 4000 in accordance with one form of the present technology may include an air filter 4110, or a plurality of air filters 4110.

In one form, an air inlet filter 4112 is located at the beginning of the pneumatic path upstream of a pressure generator 4140.

In one form, an air outlet filter 4114, for example an antibacterial filter, is located between an outlet of the pneumatic block 4020 and a patient interface 3000.

### 4.3.1.2 Muffler(s)

An RPT device 4000 in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 4.3.1.3 Pressure Generator

In one form of the present technology, a pressure generator 4140 for supplying pressurised air is a controllable blower 4142. For example, the blower 4142 may include a brushless DC motor 4144 with one or more impellers housed in a volute. The pressure generator 4140 may be capable of generating a supply or flow of air, for example at about 120 litres/minute, at a positive pressure in a range from about 4 cmH₂O to about 20 cmH₂O, or in other forms up to about 30 cmH₂O.

The pressure generator 4140 is under the control of the therapy device controller 4240.

In other forms, a pressure generator 4140 may be a piston-driven pump, a pressure regulator connected to a high-pressure source (e.g., compressed air reservoir), or a bellows.

### 4.3.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 are constructed and arranged to generate data representing respective properties of the air flow, such as a flow rate, a pressure or a temperature, at that point in the pneumatic path. The one or more transducers may also include a microphone, such as described in U.S. Pat. No. 10,773,038 B2, to measure sound within the patient circuit (e.g., tube and patient interface) for patient interface detection.

In one form of the present technology, one or more transducers 4270 are located proximate to the patient interface 3000.

In one form, a signal from a transducer 4270 may be filtered, such as by low-pass, high-pass or band-pass filtering.

### 4.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 4.3.1.6 Air Circuit

An air circuit 4170 in accordance with one aspect of the present technology is a conduit or tube constructed and arranged to allow, in use, a flow of air to travel between two components such as the pneumatic block 4020 and the patient interface 3000.

### 4.3.1.7 Oxygen Delivery

In one form of the present technology, supplemental oxygen 4180 is delivered to one or more points in the pneumatic path, such as upstream of the pneumatic block 4020, to the air circuit 4170 and/or to the patient interface 3000.

### 4.3.2 RPT Device Electrical Components

### 4.3.2.1 Power Supply

In one form of the present technology power supply 4210 is internal of the external housing 4010 of the RPT device 4000. In another form of the present technology, power supply 4210 is external of the external housing 4010 of the RPT device 4000.

In one form of the present technology power supply 4210 provides electrical power to the RPT device 4000 only. In another form of the present technology, power supply 4210 provides electrical power to both RPT device 4000 and humidifier 5000.

### 4.3.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form the input device 4220 may be constructed and arranged to allow a person to select a value and/or a menu option.

### 4.3.2.3 Central Controller

In one form of the present technology, the central controller 4230 is a processor suitable to control an RPT device 4000 such as an x86 INTEL processor.

A central controller 4230 suitable to control an RPT device 4000 in accordance with another form of the present technology includes a processor based on ARM Cortex-M processor from ARM Holdings. For example, an STM32 series microcontroller from ST MICROELECTRONICS may be used.

Another central controller 4230 suitable to control an RPT device 4000 in accordance with a further alternative form of the present technology includes a member selected from the family ARM9-based 32-bit RISC CPUs. For example, an STR9 series microcontroller from ST MICROELECTRONICS may be used.

In certain alternative forms of the present technology, a 16-bit RISC CPU may be used as the central controller 4230 for the RPT device 4000. For example, a processor from the MSP430 family of microcontrollers, manufactured by TEXAS INSTRUMENTS, may be used.

In another form of the present technology, the central controller 4230 is a dedicated electronic circuit. In another form, the central controller 4230 is an application-specific integrated circuit (ASIC). In another form, the central controller 4230 comprises discrete electronic components.

The central controller 4230 is configured to receive input signal(s) from one or more transducers 4270, one or more input devices 4220, and the humidifier 5000.

The central controller 4230 is configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300, expressed as computer programs stored in a non-transitory computer readable storage medium, such as memory 4260 or other memory described herein. In some forms of the present technology, as previously discussed, the central controller 4230 may be integrated with an RPT device 4000. However, in some forms of the present technology, some methodologies may be performed by a remotely located device or server such as the server previously mentioned. For example, the remotely located device or server may determine control settings for transfer to a ventilator or other RT device such as by detecting respiratory related events and distinguishing them by type by an analysis of stored data such as from any of the sensors described herein.

While the central controller 4230 may comprise a single controller interacting with various sensors 4270, data communications interface 4280, memory 4260, as well as other devices, the functions of controller 4230 may be distributed among more than one controller. Thus, the term “central” as used herein is not meant to limit the architecture to a single controller or processor that controls the other devices. For example, alternative architectures may include a distributed controller architecture involving more than one controller or processor, which may optionally be directly or indirectly in electronic (wired or wireless) communications with the previously described finger sensor or a server in communication with the finger sensor, such as for implementing any of the methodologies described herein. This may include, for example, a separate local (i.e., within RPT device 4000) or remotely located controller that perform some of the algorithms 4300, or even more than one local or remote memory that stores some of the algorithms. In addition, the algorithms when expressed as computer programs may comprise high level human readable code (e.g., C++, Visual Basic, other object oriented languages, etc.) or low/machine level instructions (Assembler, Verilog, etc.). Depending on the functionality of an algorithm(s), such code or instructions may be burnt in the controller, e.g., an ASIC or DSP, or be a run time executable ported to a DSP or general purpose processor that then becomes specifically programmed to perform the tasks required by the algorithm(s).

### 4.3.2.4 Clock

The RPT device 4000 may include a clock 4232 that is connected to the central controller 4230.

### 4.3.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 4.3.2.6 Protection Circuits

An RPT device 4000 in accordance with the present technology may comprise one or more protection circuits 4250.

One form of protection circuit 4250 in accordance with the present technology is an electrical protection circuit.

One form of protection circuit 4250 in accordance with the present technology is a temperature or pressure safety circuit.

### 4.3.2.7 Memory

In accordance with one form of the present technology the RPT device 4000 includes memory 4260, for example non-volatile memory. In some forms, memory 4260 may include battery powered static RAM. In some forms, memory 4260 may include volatile RAM.

Memory 4260 may be located on PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260, such as any of the memories previously described, acts as a non-transitory computer readable storage medium on which is stored computer program instructions expressing the one or more methodologies described herein, such as the one or more algorithms 4300.

### 4.3.2.8 Transducers

Transducers may be internal of the device 4000, or external of the RPT device 4000. External transducers may be located for example on or form part of the air delivery circuit 4170, e.g., at the patient interface 3000. External transducers may be in the form of non-contact sensors such as a Doppler radar movement sensor that transmit or transfer data to the RPT device 4000.

### 4.3.2.8.1 Flow Rate

A flow rate transducer 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In one example, a signal representing total flow rate Qt from the flow transducer 4274 is received by the central controller 4230.

### 4.3.2.8.2 Pressure

A pressure transducer 4272 in accordance with the present technology is located in fluid communication with the pneumatic path. An example of a suitable pressure transducer 4272 is a sensor from the HONEYWELL ASDX series. An alternative suitable pressure transducer is a sensor from the NPA Series from GENERAL ELECTRIC.

In use, a signal from the pressure transducer 4272 is received by the central controller 4230. In one form, the signal from the pressure transducer 4272 is filtered prior to being received by the central controller 4230.

### 4.3.2.8.3 Motor Speed

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 4.3.2.9 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol and may optionally communicate with any of the sensors described herein.

In one form, remote external device 4286 is one or more computers, for example a cluster of networked computers and/or server as described herein. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 4.3.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 4.3.2.10.1 Display Driver

A display driver 4292 receives as an input the characters, symbols, or images intended for display on the display 4294, and converts them to commands that cause the display 4294 to display those characters, symbols, or images.

### 4.3.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

### REFERENCES

[1] M. M. Lyons, N. Y. Bhatt, A. I. Pack, and U. J. Magalang, “Global burden of sleep-disordered breathing and its implications,” Respirology. vol. 25, no. 7, pp. 690-702, 2020, doi: https://doi.org/10.1111/reso 13838.
[2] N. A. Dewan, F. J. Nieto, and V. K. Somers, “Intermittent hypoxemia and OSA: implications for comorbidities,” (in eng), Chest. vol. 147, no. 1, pp. 266-274, January 2015, doi: 10.1378/chest.14-0500.
[3] P. Lévy et al., “Obstructive sleep apnoea syndrome,” Nature Reviews Disease Primers. vol. 1, no. l. p. 15015, 2015 Jun. 25 2015, doi: 10.1038/nrdp.2015.15.
[4] S. Garbarino, W. A. Bardwell, O. Guglielmi, C. Chiorri, E. Bonanni, and N. Magnavita, “Association of Anxiety and Depression in Obstructive Sleep Apnea Patients: A Systematic Review and Meta-Analysis,” Behavioral Sleep Medicine. vol. 18, no. 1, pp. 35-57, 2020 Jan. 2 2020, doi: 10.1080/15402002.2018.1545649.
[5] V. Brunetti et al . . . “Association of Obstructive Sleep Apnea and Atrial Fibrillation in Acute Ischemic Stroke: A Cross-Sectional Study,” J Pers Med. vol. 13, no. 3, Mar. 15 2023, doi: 10.3390/jpm 13030527.
[6] X. Li et al., “Associations of Sleep-disordered Breathing and Insomnia with Incident Hypertension and Diabetes. The Hispanic Community Health Study/Study of Latinos,” American Journal of Respiratory and Critical Care Medicine. vol. 203, no. 3, pp. 356-365, 2021, doi: 10.1164/rccm.201912-23300C.
[7] P. Macek et al., “Obstructive Sleep Apnea as a Predictor of a Higher Risk of Significant Coronary Artery Disease Assessed Non-Invasively Using the Calcium Score,” Life (Basel). vol. 13, no. 3, Mar. 1 2023, doi: 10.3390/life13030671.
[8] J. Streatfeild, J. Smith, D. Mansfield, L. Pezzullo, and D. Hillman, “The social and economic cost of sleep disorders,” Sleep. vol. 44, no. 11, p. zsab 132, 2021.
[9] S. P. Patil, I. A. Ayappa, S. M. Caples, R. J. Kimoff, S. R. Patel, and C. G. Harrod. “Treatment of Adult Obstructive Sleep Apnea With Positive Airway Pressure: An American Academy of Sleep Medicine Systematic Review, Meta-Analysis, and GRADE Assessment,” J Clin Sleep Med. vol. 15, no. 2, pp. 301-334, Feb. 15 2019, doi: 10.5664/jcsm.7638.
[10] C. Sullivan, M. Berthon-Jones, F. Issa, and L. Eves, “Reversal of obstructive sleep apnoca by continuous positive airway pressure applied through the nares,” The Lancet. vol. 317, no. 8225, pp. 862-865. 1981.
[11] A. Gharib, “Effect of continuous positive airway pressure on the respiratory system: a comprehensive review,” The Egyptian Journal of Bronchology. vol. 17, no. 1, p. 1, 2023 Jan. 3 2023, doi: 10.1186/s43168-022-00175-1.
[12] ResMed. “CPAP life expectancy: How long do CPAP machines last?” https://www.resmed.com/en-us/sleep-apoco/sleep-blog/cpap-life-expectanex-how-long-docpap-machines-last/(accessed Mar. 11, 2023, 2023).
[13] O. Yetkin, E. Kunter, and H. Gunen, “CPAP compliance in patients with obstructive sleep apnea syndrome,” Sleep and Breathing. vol. 12, no. 4, pp. 365-367, 2008 Nov. 1 2008, doi: 10.1007/s11325-008-0188-4.
[14] S. Parthasarathy, “Mask interface and CPAP adherence,” Journal of Clinical Sleep Medicine. vol. 4, no. 5, pp. 511-512, 2008.
[15] M. Ghadiri and R. R. Grunstein, “Clinical side effects of continuous positive airway pressure in patients with obstructive sleep apnoca.” Respirology. vol. 25, no. 6, pp. 593-602, 2020.
[16] C. Chen, J. Wang. L. Pang. Y. Wang. G. Ma, and W. Liao, “Telemonitor care helps CPAP compliance in patients with obstructive sleep apnea: a systemic review and meta-analysis of randomized controlled trials,” Therapeutic advances in chronic disease. vol. 11, p. 2040622320901625, 2020.
[17] A. M. Sawyer, N. S. Gooneratne, C. L. Marcus, D. Ofer, K. C. Richards, and T. E. Weaver, “A systematic review of CPAP adherence across age groups: Clinical and empiric insights for developing CPAP adherence interventions,” Sleep Medicine Reviews. vol. 15, no. 6, pp. 343-356, 2011 Dec. 1/2011, doi: https://doi.org/10.1016/snurx 2011.01.003
[18] R. G. Hooper, “CPAP Therapeutic Options for Obstructive Sleep Apnea.” Pragmatic and Observational Research. vol. 11, no. null, pp. 67-76, 2020 Jul. 13 2020, doi: 10.2147/POR.S258632.
[19] L.-Y. Chen et al., “In search of a better CPAP interface: A network meta-analysis comparing nasal masks, nasal pillows and oronasal masks,” Journal of Sleep Research. vol. 31, no. 6, p. e13686, 2022, doi: https://doi org/10.1111/sr. 13686.
[22] D. C. M. L. Holley, S. P. Farrugia, “Acoustic detection for respiratory treatment apparatus,” US Patent Appl. U.S. Pat. No. 13,148,730, 2010.
[23] A. E. Maxwell. T. A. Warner, and F. Fang, “Implementation of machine-learning classification in remote sensing: an applied review,” International Journal of Remote Sensing. vol. 39, no. 9. pp. 2784-2817. 2018 May 3 2018, doi: 10.1080/01431161.2018.1433343.
[24] E. M. T. Semen, “System and method for medical device identifier,” US Patent U.S. Pat. No. 20,200,143915A1 Patent Appl. U.S. Pat. No. 16,572,870, 2020 May 7. 2017.
[25] J. Tolson et al., “Filters Alter the Performance of Noninvasive Ventilators,” Respiratory Care. vol. 67, no. 7, pp. 795-800, 2022, doi: 10.4187/respcare.09365.
[26] D. W. S. B.I. Shelly. R.A. Sofranko, L.A. Jacob, J.R. Pujol, “WIRELESS IDENTIFICATION OF A COMPONENT OF A PRESSURE SUPPORT SYSTEM,” EU, 2017.
[27] X. Han et al., “Novel resonant pressure sensor based on piezoresistive detection and symmetrical in-plane mode vibration,” Microsystems & Nanoengineering. vol. 6, no. 1, p. 95, 2020 Nov. 16 2020, doi: 10.1038/s41378-020-00207-0.
[28] A. J. Klosinski and C. R. Willcox, “Resonant frequency based pressure sensor,” (in en), Patent EP2553415A1 Patent Appl. EP11707979A. 2013 Feb. 6, 2013. [Online]. Available: https://patents.google.com/patent/EP25534ISA Ion
[29] D. C. M. L Holley, S.P. Farrugia, “Acoustic Detection for Respiratory Treatment Apparatus,” US Patent U.S. Pat. No. 10,773,038B2, 2020.
[30] W. A. Yost, “Fundamentals of hearing: An introduction,” ed: Acoustic Society of America, 2001.
[31] R. D. Luce, Sound & Hearing: a conceptual introduction. Psychology Press, 2013.
[32] N. R. C. U. C. o. D. D. f. I. w. H. Impairments:, Dobie RA, and e. Van Hemel S, Basics of Sound. the Ear. and Hearing-Hearing loss: Determining eligibility for social security benefits. 2004.
[33] E. Zwicker, “Subdivision of the Audible Frequency Range into Critical Bands (Frequenzgruppen),” The Journal of the Acoustic Society of America. vol. 33, no. 2, pp. 248-248, 2005, doi: 10.1121/1.1908630.
[34] R. T. Beyer and D. R. Raichel, “Sounds of our times, two hundred years of acoustics,” The Journal of the Acoustic Society of America. vol. 106, no. 1, pp. 15-16, 1999, doi: 10.1121/1.428229.
[35] R. Merino-Martinez et al., “A review of acoustic imaging methods using phased microphone arrays.” CEAS Aeronautical Journal, vol. 10, no. 1, pp. 197-230, 2019 Mar. 1 2019, doi: 10.1007/s13272-019-00383-4
[36] R. Ghaffarivardavagh, J. Nikolajczyk, S. Anderson, and X. Zhang, “Ultra-open acoustic metamaterial silencer based on Fano-like interference,” Physical Review B. vol. 99, no. 2. p. 024302, Jan. 4, 2019, doi: 10.1103/PhysRevB.99.024302.
[37] D. R. Griffin, “Echolocation by Blind Men, Bats and Radar,” Science, vol. 100, no. 2609, pp. 589-590, 1944, doi: doi: 10.1126/science. 100.2609.589.
[38] G. Jones, “Echolocation,” Curr Biol. vol. 15, no. 13, pp. R484-8, Jul. 12 2005, doi: 10.1016/j.cub.2005.06.051.
[39] G. W. Pierce and D. R. Griffin, “Experimental Determination of Supersonic Notes Emitted by Bats.” Journal of Mammalogy. vol. 19, no. 4, pp. 454-455, 1938, doi: 10.2307/1374231.
[40] B. N. Schenkman and M. E. Nilsson, “Human Echolocation: Blind and Sighted Persons' Ability to Detect Sounds Recorded in the Presence of a Reflecting Object.” Perception. vol. 39, no. 4, pp. 483-501, 2010. doi: 10.1068/p6473.
[41] T. A. Stroffregen and J. B. Pittenger, “Human Echolocation as a Basic Form of Perception and Action,” Ecological Psychology. vol. 7, no. 3, pp. 181-216, 1995 Sep. 1 1995, doi: 10.1207/s15326969cco0703_2.
[42] A. P. Sarvazyan, M. W. Urban, and J. F. Greenleaf, “Acoustic Waves in Medical Imaging and Diagnostics,” Ultrasound in Medicine & Biology. vol. 39, no. 7, pp. 1133-1146, 2013 Jul. 1/2013, doi: https://doi.org/10.1016/altrasmedbio 2013 02 006.
[43] “Gestational age determination of ultrasound foetal images using artificial neural network,” International Journal of Bioinformatics Research and Applications. vol. 18, no. 1-2, pp. 113129, 2022, doi: 10.1504/ijbra.2022.121766.
[44] M. Rivas-Lasarte et al., “Lung ultrasound-guided treatment in ambulatory patients with heart failure: a randomized controlled clinical trial (LUS-HF study),” European journal of heart failure. vol. 21, no. 12, pp. 1605-1613, 2019.
[45] V. Chan and A. Perlas, “Basics of Ultrasound Imaging.” in Atlas of Ultrasound-Guided Procedures in Interventional Pain Management, S. N. Narouze Ed. New York, NY: Springer New York, 2011, pp. 13-19.
[46] T. G. Leighton, “What is ultrasound?,” Progress in Biophysics and Molecular Biology. vol. 93, no. 1, pp. 3-83, 2007 Jan. 1/2007, doi: https://doi.on/10 1016/pbiomolbo 2006 07 026.
[47] M. W. Schellenberg and H. K. Hunt, “Hand-held optoacoustic imaging: A review,” Photoacoustics. vol. 11, pp. 14-27, 2018 Sep. 1/2018, doi: https://doi.org/10 10164 pacs.2018.07.001.
[48] K. U. Paul and P. Manojit, “Recent advances toward preclinical and clinical translation of photoacoustic tomography: a review,” Journal of Biomedical Optics. vol. 22, no. 4, p. 041006, 11/1 2016, doi: 10.1117/1.JBO.22.4.041006.
[49] X. L. Dean-Ben, T. F. Fehm, S. J. Ford, S. Gottschalk, and D. Razansky, “Spiral volumetric optoacoustic tomography visualizes multi-scale dynamics in mice,” Light: Science & Applications. vol. 6, no. 4. pp. c16247-c16247, 2017 Apr. 1 2017, doi: 10.1038/lsa.2016.247.
[50] B. M. Howe, J. Miksis-Olds, E. Rehm, H. Sagen, P. F. Worcester, and G. Haralabus, “Observing the oceans acoustically,” Frontiers in Marine Science. vol. 6, p. 426, 2019.
[51] M. Johnson, N. A. de Soto, and P. T. Madsen, “Studying the behaviour and sensory ecology of marine mammals using acoustic recording tags: a review,” Marine Ecology Progress Series. vol. 395. pp. 55-73, 2009.
[52] W. Munk and C. Wunsch, “Ocean acoustic tomography: a scheme for large scale monitoring.” Deep Sea Research Part A. Oceanographic Research Papers, vol. 26, no. 2, pp. 123-161, 1979 Feb. 1/1979, doi: https://dot org/10.1016/0198-0149 (79) 90073-6.
[53] C. Othmani et al., “Acoustic tomographic reconstruction of temperature and flow fields with focus on atmosphere and enclosed spaces: A review,” Applied Thermal Engineering. vol. 223, p. 119953, 2023 Mar. 25/2023. doi: https://doi org/10.1016/.applthermaleng.2022.119953
[54] F. Li, X. Yang. Y. Zhang. W. Luo, and W. Gan, “Passive ocean acoustic tomography in shallow water,” The Journal of the Acoustic Society of America. vol. 145, no. 5, pp. 28232830, 2019.
[55] B. P. Bogert, “The quefrency alanysis of time series for echoes: Cepstrum, pseudoautocovariance, cross-cepstrum and saphe cracking.” in Proc. Symposium Time Series Analysis. 1963, 1963, pp. 209-243.
[56] A. V. Oppenheim and R. W. Schafer, “From frequency to quefrency: a history of the cepstrum,” IEEE Signal Processing Magazine. vol. 21, no. 5, pp. 95-106, 2004, doi: 10.1109/MSP.2004.1328092.
[57] R. B. Randall. “A history of cepstrum analysis and its application to mechanical problems.” Mechanical Systems and Signal Processing. vol. 97. pp. 3-19. 2017 Dec. 1/2017. doi: https/donors/10.1016/smossp 2016.12.026
[58] T. Drugman. B. Bozkurt, and T. Dutoit. “Complex cepstrum-based decomposition of speech for glottal source estimation.” arXiv preprint arXiv: 1912.12602. 2019.
[59] M. El Badaoui. F. Guillet, and J. Daniere. “New applications of the real cepstrum to gear signals. including definition of a robust fault indicator.” Mechanical Systems and Signal Processing. vol. 18. no. 5. pp. 1031-1046. 2004.
[60] T.-C. Che. H.-F. Duan, and P. J. Lec. “Transient wave-based methods for anomaly detection in fluid pipes: A review.” Mechanical Systems and Signal Processing. vol. 160. p. 107874. 2021 Nov. 1/2021. doi: https://doi.org/10 1016/smssp 2021 107874.
[61] R. B. Randall. “Cepstrum Analysis and Gearbox Fault Diagnosis.” 1976.
[62] H. Li. Y. Zhang. and H. Zheng. “Gear fault detection and diagnosis under speed-up condition based on order cepstrum and radial basis function neural network.” Journal of Mechanical Science and Technology. vol. 23. no. 10. pp. 2780-2789. 2009 Oct. 1 2009. doi: 10.1007/s12206-009-0730-8.
[63] S. Ziaran and R. Darula. “Determination of the State of Wear of High Contact Ratio Gear Sets by Means of Spectrum and Cepstrum Analysis.” Journal of Vibration and Acoustics. vol. 135. no. 2. 2013. doi: 10.1115/1.4023208.
[64] Y.-C. Choi and Y.-H. Kim, “Fault detection in a ball bearing system using minimum variance cepstrum.” Measurement Science and Technology. vol. 18. no. 5. p. 1433. 2007 Mar. 20 2007. doi: 10.1088/0957-0233/18/5/031.
[65] Y. Liu. Z. Jiang. H. Haizhou, and J. Xiang. “Asymmetric penalty sparse model based cepstrum analysis for bearing fault detections.” Applied Acoustics. vol. 165. p. 107288. 2020 Aug. 1/2020. doi: https://doi.org/10.1016/apacoust.2020.107288.
[66] C. Peeters. P. Guillaume, and J. Helsen. “A comparison of cepstral editing methods as signal pre-processing techniques for vibration-based bearing fault detection.” Mechanical Systems and Signal Processing. vol. 91. pp. 354-381. 2017 Jul. 1/2017. doi: https://doi. org/10 1016/j.ymssp.2016 12.036.
[67] P. Yao et al . . . “Intelligent rolling bearing imbalanced fault diagnosis based on Mel-Frequency Cepstrum Coefficient and Convolutional Neural Networks.” Measurement. vol. 205. p. 112143. 2022 Dec. 1/2022. doi: https://doi.org/10 1016/measurement 2022 112143.
[68] S. Majumder. S. Pal, and P. K. Dutta. “A Comparative Study for Disease Identification from Heart Auscultation using FFT. Cepstrum and DCT Correlation Coefficients.” in 13th International Conference on Biomedical Engineering. Berlin, Heidelberg. C. T. Lim and J. C. H. Goh. Eds . . . 2009//2009: Springer Berlin Heidelberg. pp. 754-757.
[69] E.-S. A. El-Dahshan and M. M. Bassiouni. “Intelligent methodologies for cardiac sound signals analysis and characterization in cepstrum and time-scale domains.” Computational Intelligence. vol. 36, no. 2. pp. 427-458. 2020. doi: https://doi.org/10.1111/coin. 12244.
[70] P. Castiglioni. G. Parati, and A. Faini. “Cepstral Analysis for Scoring the Quality of Electrocardiograms for Heart Rate Variability.” (in eng). Front Physiol. vol. 13. p. 921210. 2022. doi: 10.3389/fphys.2022.921210.
[71] S. Martin-González, A. G. Ravelo-García. J. L. Navarro-Mesa. and E. Hernández-Pérez. “Combining Heart Rate Variability and Oximetry to Improve Apneic Event Screening in Non-Desaturating Patients.” Sensors. vol. 23, no. 9. p. 4267. 2023. [Online]. Available: https://www.mndpi.com/1424-8220/23/9/4267.
[72] C. T. Chuang, T. Chang, Y. T. Chiang, and F. R. Chang, “Adaptive filtering for heart rate estimation using cepstrum technique,” in 2016 International Conference on System Science and Engineering (ICSSE). 7-9 Jul. 2016 2016, pp. 1-3, doi: 10.1109/ICSSE.2016.7551622.
[73] Y. Zhu et al., “Heart rate estimation from FBG sensors using cepstrum analysis and sensor fusion,” in 2014 36th annual international conference of the IEEE engineering in medicine and biology society, 2014: IEEE, pp. 5365-5368.
[74] I. Sadek, J. Biswas, and B. Abdulrazak, “Ballistocardiogram signal processing: a review,” (in eng), Health Inf Sci Syst. vol. 7, no. 1, p. 10, December 2019, doi: 10.1007/s13755-019-00717.
[75] S. Razvarz. R. Jafari, and A. Gegov, “A Review on Different Pipeline Defect Detection Techniques,” in Flow Modelling and Control in Pipeline Systems: A Formal Systematic Approach, S. Razvarz, R. Jafari, and A. Gegov Eds. Cham: Springer International Publishing, 2021, pp. 25-57.
[76] J. D. Shucksmith, J. B. Boxall, W. J. Staszewski, A. Seth, and S. B. Beck, “Onsite leak location in a pipe network by cepstrum analysis of pressure transients,” Journal-American Water Works Association. vol. 104, no. 8, pp. E457-E465, 2012.
[77] N. Motazedi and S. Beck, “Leak detection using cepstrum of cross-correlation of transient pressure wave signals,” Proceedings of the Institution of Mechanical Engineers. Part C: Journal of Mechanical Engineering Science. vol. 232, no. 15, pp. 2723-2735, 2018, doi: 10.1177/0954406217722805.
[78] R. Santos, S. d. A. W. F. V. Silva, S. Cruz, and A. M. Fileti, “Spectral Analysis for Detection of Leaks in Pipes Carrying Compressed Air.” (in en), Chemical Engineering Transactions, vol. 32, pp. 1363-1368, 2013 Jun. 20/2013, doi: 10.3303/CET1332228.
[79] R. Worley et al., “Robot Localization in Water Pipes Using Acoustic Signals and Pose Graph Optimization,” Sensors (Basel). vol. 20, no. 19, Sep. 29 2020, doi: 10.3390/s20195584.
[80] R. Worley, Y. Yu, and S. Anderson, “Acoustic Echo-Localization for Pipe Inspection Robots.” in 2020 IEEE International Conference on Multisensor Fusion and Integration for Intelligent Systems (MFI), 14-16 Sep. 2020 2020, pp. 160-165. doi: 10.1109/MFI49285.2020.9235225. [Online]. Available: https://iecexplore.jcce org/document/9235225/
[81] Y. Yu, R. Worley, S. Anderson, and K. V. Horoshenkov, “Microphone array analysis for simultaneous condition detection, localization, and classification in a pipe,” The Journal of the Acoustic Society of America. vol. 153, no. 1, pp. 367-383, 2023 Jan. 1 2023, doi: 10.1121/10.0016856.
[82] M. Taghvaei, S. B. M. Beck, and W. J. Staszewski, “Leak detection in pipelines using cepstrum analysis.” Measurement Science and Technology. vol. 17, pp. 367-372, Feb. 1, 2006 2006, doi: 10.1088/0957-0233/17/2/018.
[83] M. Taghvaei, S. Beck, and J. Boxall, “Leak Detection in Pipes Using Induced Water Hammer Pulses and Cepstrum Analysis,” International Journal of COMADEM. vol. 13, no. 1, 2010. https://joumals.sagepub.com/doi/10 1177/0954406217722805.
[84] X.-J. Wang. M. F. Lambert, A. R. Simpson, J. A. Liggett, and J. P. V 1′ tkovsky, “Leak detection in pipelines using the damping of fluid transients,” Journal of Hydraulic Engineering. vol. 128, no. 7, pp. 697-711, 2002.
[85] P. J. Lee, M. F. Lambert, A. R. Simpson, J. P. Vítkovsky, and J. Liggett, “Experimental verification of the frequency response method for pipeline leak detection,” Journal of Hydraulic research. vol. 44, no. 5. pp. 693-707, 2006.
[86] J. Gong. M. F. Lambert. A. R. Simpson, and A. C. Zecchin. “Single-Event Leak Detection in Pipeline Using First Three Resonant Responses.” Journal of Hydraulic Engineering. vol. 139. no. 6. pp. 645-655. 2013. doi: doi: 10.1061/(ASCE) HY.1943-7900.0000720.
[87] T.-C. Che. H. Duan, P. J. Lec. B. Pan, and M. Ghidaoui, “Transient frequency responses for pressurized water pipelines containing blockages with linearly varying diameters.” Journal of Hydraulic Engineering. vol. 144. no. 8. p. 04018054. 2018.
[88] S. Fox. J. Boxall, and R. Collins. “Derivation and validation of a leakage model for longitudinal slits in polyethylene pipes.” Journal of Hydraulic Engineering. vol. 144. no. 7. p. 04018034. 2018.
[89] L. E. Kinsler. A. R. Frey. A. B. Coppens, and J. V. Sanders, Fundamentals of acoustics. John wiley & sons. 2000.
[90] N. Hiremath. V. Kumar. N. Motahari, and D. Shukla. “An Overview of Acoustic Impedance Measurement Techniques and Future Prospects.” Metrology. vol. 1. no. 1. pp. 17-38. 2021. [Online]. Available: https://www.mdpi.com/2673-8244/1/1/2.
[91] F. Fahy. “Rapid method for the measurement of sample acoustic impedance in a standing wave tube.” Journal of Sound Vibration. vol. 97. no. 1. pp. 168-170. 1984.
[92] X. Duanqi. W. Zheng. and C. Jinjing. “Acoustic design of a reverberation chamber.” Applied Acoustics. vol. 32. no. 2. 1991. doi: pp. 83-91. 1991 Jan. 1/https://doi.org/10.1016/0003682X (91) 90050-0.
[93] P. M. Morse. R. H. Bolt. and R. L. Brown. “Acoustic Impedance and Sound Absorption.” The Journal of the Acoustic Society of America. vol. 12. no. 2. pp. 217-227. 2005. doi: 10.1121/1.1916094.
[94] T. W. Bartel. “Effect of absorber geometry on apparent absorption coefficients as measured in a reverberation chamber.” The Journal of the Acoustic Society of America. vol. 69. no. 4. pp. 1065-1074. 1981. doi: 10.1121/1.385685.
[95] A. F. Seybert and D. F. Ross. “Experimental determination of acoustic properties using a two-microphone random-excitation technique.” The Journal of the Acoustic Society of America. vol. 61. no. 5. pp. 1362-1370. 1977. doi: 10.1121/1.381403.
[96] J. Y. Chung and D. A. Blaser. “Transfer function method of measuring in-duct acoustic properties. II. Experiment.” The Journal of the Acoustic Society of America. vol. 68. no. 3. pp. 914-921. 1980. doi: 10.1121/1.384779.
[97] J. Y. Chung and D. A. Blaser. “Transfer function method of measuring in-duct acoustic properties. I. Theory.” The Journal of the Acoustic Society of America. vol. 68. no. 3. pp. 907-913. 1980. doi: 10.1121/1.384778.
[98] W. T. Chu. “Transfer function technique for impedance and absorption measurements in an impedance tube using a single microphone.” The Journal of the Acoustic Society of America. vol. 80. no. 2. pp. 555-560. 1986, doi: 10.1121/1.394050.
[99] D. H. Keefe. “Physical modeling of wind instruments.” Computer Music Journal, vol. 16. no. 4. pp. 57-73. 1992.
[100] K. R. Lutchen. K. Yang. D. W. Kaczka. and B. Suki. “Optimal ventilation waveforms for estimating low-frequency respiratory impedance.” Journal of applied physiology. vol. 75. no. 1. pp. 478-488. 1993.
[101] J. J. Pillow. “High-frequency oscillatory ventilation: mechanisms of gas exchange and lung mechanics.” Critical care medicine. vol. 33. no. 3. pp. S135-S141. 2005.
[102] F. E. White. “Fundamentals of Acoustics by Lawrence E. Kinsler. Austin R. Frey. Alan B. Coppens. and James V. Sanders.” The Journal of the Acoustic Society of America. vol. 72. no. 3. pp. 1090-1090. 1982. doi: 10.1121/1.388211.
[103] Q. Meng. J. Zhang, J. Kang, and Y. Wu, “Effects of sound environment on the sleep of college students in China.” Science of The Total Environment. vol. 705, p. 135794, 2020 Feb. 25/2020, doi: https://doi.org/10.1016/scitotony 2019.135794.
[104] T. X. Vuong and A. M. Al-Jumaily, “An investigation into the noise generation of CPAP devices: Experimental and numerical study.” Applied Acoustics. vol. 114, pp. 281-293, 2016 Dec. 15/2016, doi: https://doi.org/10 1016/apacoust.2016.08.005.
[105] M. Scheel, T. Schauer, A. Berndt, and O. Simanski, “Model-based control approach for a CPAP-device considering patient's breathing effort.” IFAC-PapersOnLine. vol. 50, no. 1, pp. 9948-9953, 2017 Jul. 1/2017. doi: https://doi org/10.1016/j.ifacol 2017.08 1572.
[106] T. X. Vuong. A. Al-Jumaily, and R. Paxton, “Computational Analysis of the Dynamics Flow in the Duct System of an Continuous Positive Airways Pressure Device,” in ASME 2020 International Mechanical Engineering Congress and Exposition, 2020, vol. Volume 5: Biomedical and Biotechnology, V005T05A028, doi: 10.1115/imece2020-23333. [Online]. Available: https://doi.org/10.1115/MECE2020-23333
[107] D. Wolfram and T. H. Carolus, “Experimental and numerical investigation of the unsteady flow field and tone generation in an isolated centrifugal fan impeller,” Journal of Sound and Vibration, vol. 329. no. 21, pp. 4380-4397, 2010 Oct. 11/2010, doi: https/dot org/10.10166 jsy 2010.04.034.
[108] B. Rajavel and M. G. Prasad, “Acoustics of Corrugated Pipes: A Review,” Applied Mechanics Reviews. vol. 65, no. 5, 2013, doi: 10.1115/1.4025302.
[109] M. Popescu, S. T. Johansen, and W. Shyy, “Flow-Induced Acoustics in Corrugated Pipes.” Commun. Comput. Phys. vol. 10, 2010.
[110] J. Elliott, “Corrugated pipe flow,” Lecture Notes on the Mathematics of acoustics. pp. 207222, 2005.
[111] S. Serafin and J. Kojs, “Computer models and compositional applications of plastic corrugated tubes,” Organised Sound. vol. 10, no. 1, pp. 67-73, 2005.
[112] A. M. Binnie, “Self-induced waves in a conduit with corrugated walls. II. Experiments with air in corrugated and finned tubes,” Proceedings of the Royal Society of London. Series A. Mathematical and Physical Sciences, vol. 262, no. 1309, pp. 179-191, 1961.
[113] FDA, “Medical Devices: Anesthesiology Devices; Classification of the Positive Airway Pressure Delivery System,” Food and Drug Administration, Rule Oct. 19, 2018 2018. [Online]. Available: https://says federalregister gov/doct ments/2018/10/19/2018-22840/medical vices-anesthesiology-devices-classification-of-the-positive-a airway pressure-delivery
[114] G. Nilius, U. Domanski, M. Schroeder, H. Wochrle, A. Graml, and K.-J. Franke, “Mask humidity during CPAP: influence of ambient temperature, heated humidification and heated tubing.” Nature and Science of Sleep. vol. 10, pp. 135-142, 2018 Dec. 31 2018, doi: 10.2147/NSS.S158856.
[115] C. Loudon and K. McCulloh, “Application of the Hagen-Poiseuille Equation to Fluid Feeding through Short Tubes,” Annals of the Entomological Society of America. vol. 92, no. 1, pp. 153-158, 1999, doi: 10.1093/aesa/92.1.153.
[116] G. O. Brown, “The History of the Darcy-Weisbach Equation for Pipe Flow Resistance,” (in En), pp. 34-43, 2012 Apr. 26/2012. doi: 10.1061/40650 (2003) 4.
[117] C. A. Massie, R. W. Hart, K. Peralez, and G. N. Richards, “Effects of humidification on nasal symptoms and compliance in sleep apnea patients using continuous positive airway pressure.” Chest. vol. 116, no. 2, pp. 403-8, August 1999, doi: 10.1378/chest.116.2.403.
[118] O. Cramer, “The variation of the specific heat ratio and the speed of sound in air with temperature, pressure, humidity, and CO₂ concentration,” The Journal of the Acoustic Society of America. vol. 93, no. 5, pp. 2510-2516, 1993, doi: 10.1121/1.405827.
[120] D. C. Toledo-Pérez, J. Rodríguez-Reséndiz, R. A. Gómez-Loenzo, and J. C. Jauregui-Correa, “Support Vector Machine-Based EMG Signal Classification Techniques: A Review,” Applied Sciences. vol. 9, no. 20, p. 4402, 2019. [Online]. Available: https://wwwwww.mdpi.com/2076-3417/9/20/4402
[121] S. Celin and K. Vasanth, “ECG Signal Classification Using Various Machine Learning Techniques,” Journal of Medical Systems. vol. 42, no. 12, p. 241, 2018 Oct. 18 2018, doi: 10.1007/s10916-018-1083-6.
[122] StrataSys, “SAFETY DATA SHEET DraftGrey,” StrataSys, Online, Safety Data Sheet 02.02.23 2023. [Online]. Available: https://support.stratasss.com/en/Materials/Pol Jet/DraftGrey
[123] NemArt, “TV Static Noise 10 hours, HD 1080p,” ed.
[124] M. Ghazali, S. Beck, J. Shucksmith, J. Boxall, and W. Staszewski, “Comparative study of instantaneous frequency based methods for leak detection in pipeline networks,” Mechanical Systems and Signal Processing. vol. 29, pp. 187-200, 2012.
[125] S. M. Riedy, M. G. Smith, S. Rocha, and M. Basner, “Noise as a sleep aid: A systematic review,” Sleep medicine reviews. vol. 55, pp. 101385-101385, 2021, doi: 10.1016/j.smrv.2020.101385.
[126] B. Jurado-Gamez, O. Guglielmi, F. Gude-Sampedro, and G. Buela-Casal, “Effect of CPAP therapy on job productivity and psychosocial occupational health in patients with moderate to severe sleep apnea,” Sleep and Breathing. vol. 19, no. 4, pp. 1293-1299, 2015 Dec. 1 2015, doi: 10.1007/s11325-015-1162-6.
[127] E. Avlonitou, F. Kapsimalis, G. Varouchakis, C. I. Vardavas, and P. Behrakis, “Adherence to CPAP therapy improves quality of life and reduces symptoms among obstructive sleep apnea syndrome patients,” Sleep and Breathing. vol. 16, no. 2, pp. 563-569, 2012 Jun. 1 2012, doi: 10.1007/s11325-011-0543-8.
[128] M.-C. Rotty et al., “Mask side-effects in long-term CPAP-patients impact adherence and sleepiness: the Interface Vent real-life study,” Respiratory Research. vol. 22, no. 1, p. 17, 2021 Jan. 15 2021, doi: 10.1186/s12931-021-01618-x.
[129] G. Labarca, D. Saavedra, J. Dreyse, J. Jorquera, and F. Barbe, “Efficacy of CPAP for Improvements in Sleepiness, Cognition, Mood, and Quality of Life in Elderly Patients With OSA: Systematic Review and Meta-analysis of Randomized Controlled Trials,” Chest. vol. 158, no. 2, pp. 751-764, 2020 Aug. 1/2020, doi: https://doi.org/10 1016/.chest.2020.03.049.

1. A patient interface model for delivering a respiratory therapy to a patient via an air circuit from a respiratory therapy device, the patient interface model comprising:
an inlet that is connectable with the air circuit;
a plenum chamber integrated with the inlet and in fluid communication with the inlet, wherein the inlet and plenum chamber form a flow path for the respiratory therapy, wherein the plenum chamber is configured for coupling with a respiratory system of the patient for delivery of the respiratory therapy to the patient; and
a positioning and stabilising structure configured to engage the plenum chamber with the patient,
wherein the patient interface is adapted with one or more first model specific acoustical features in the flow path that are configured to produce an acoustic response to sound in the flow path that distinguishes the model of the patient interface from different patient interface models that have respectively different one or more model specific acoustical features.

2. The patient interface model of claim 1, wherein the one or more first model specific acoustical features comprises a difference in material composition of at least a portion of a wall section of the flow path.

3. The patient interface model of claim 2, wherein the difference in material composition comprises a preceding section and an interceding section, each formed of a different material composition to the other.

4. The patient interface model of claim 3, wherein a repeated sequence of the preceding section and the interceding section form a plurality of interceding sections.

5. The patient interface model of claim 4, wherein the one or more first model specific acoustical features comprises a count of the plurality of interceding sections that is different from a count of a plurality of interceding sections of the different patient interface models that have respectively different one or more model specific acoustical features.

6. The patient interface model of claim 2, wherein the difference in material composition comprises a first material composition that is different from a second material composition of the different patient interface models that have respectively different one or more model specific acoustical features.

7. The patient interface model of claim 1, wherein the one or more first model specific acoustical features comprises a length of a portion of the flow path that is different from lengths of the portion of the flow path of the different patient interface models that have respectively different one or more model specific acoustical features.

8. The patient interface model of claim 7, wherein the portion of the flow path comprises a sounding chamber.

9. The patient interface model of claim 8, wherein the sounding chamber comprises an expansion of the flow path.

10. The patient interface model of claim 7, wherein the length of the portion of the flow path that is different from lengths of the portion of the flow path of the different patient interface models is different by at least five millimeters.

11. The patient interface model of claim 10, wherein each length of the lengths of the portion of the flow path of the different patient interface models are different from each other by at least five millimeters.

12. The patient interface model of claim 1, wherein the one or more first model specific acoustical features comprises a first deviation or gradient in a portion of the flow path.

13. The patient interface model of claim 12, wherein the first deviation or gradient comprises a change in diameter in the portion of the flow path.

14. The patient interface model of claim 12, wherein the one or more first model specific acoustical features comprises a second deviation or gradient in the portion of the flow path.

15. The patient interface model of claim 14, wherein the first deviation or gradient and the second deviation or gradient form a sounding chamber having a first length that is different from lengths of sounding chambers in flow paths of the different patient interface models that have respectively different one or more model specific acoustical features.

16. The patient interface model of claim 15, wherein additional deviations or gradients form a further sounding chamber having a second length that is different from lengths of further sounding chambers in flow paths of the different patient interface models that have respectively different one or more model specific acoustical features.

17. The patient interface model of claim 16, wherein the first length is different from the second length by at least five millimeters.

18. The patient interface model of claim 1, wherein the one or more first model specific acoustical features comprises a difference in material thickness of at least a portion of a wall section of the flow path.

19. The patient interface model of claim 18, wherein the difference in material thickness comprises a preceding section and an interceding section, each formed of a different material thickness to the other.

20. The patient interface model of claim 19, wherein a repeated sequence of the preceding section and the interceding section form a plurality of interceding sections.

21. The patient interface model of claim 20, wherein the one or more first model specific acoustical features comprises a count of the plurality of interceding sections that is different from a count of a plurality of interceding sections of the different patient interface models that have respectively different one or more model specific acoustical features.

22. The patient interface model of claim 17, wherein the difference in material composition comprises a first material thickness that is different from a second material thickness of the different patient interface models that have respectively different one or more model specific acoustical features.

23. The patient interface model of claim 1, wherein the one or more first model specific acoustical features in the flow path are formed in the inlet.

24. The patient interface model of claim 23, wherein the inlet comprises a swivel.

25. The patient interface model of claim 1, wherein an inner surface of the air circuit from the respiratory therapy device is substantially smooth.
